(19) United States
(12) Patent Application Publication
(10) Pub. No.: US 2005/0192339 A1
(43) Pub. Date: Sep. 1, 2005

(54) Compounds

(76) Inventors: Andrew Browning, Knivsta (SE); Martin Scobie, Uppsala (SE)

Correspondence Address: FISH & RICHARDSON PC, 225 FRANKLIN ST, BOSTON, MA, 02110, US

(21) Appl. No.: 10/995,948

(22) Filed: Nov. 23, 2004

Related U.S. Application Data
(60) Provisional application No. 60/549,644, filed on Mar. 3, 2004.

(30) Foreign Application Priority Data
Nov. 26, 2003 (SE) 0303181-2

(51) Int. Cl. C07D 209/04; A61K 31/405

(52) U.S. Cl. 514/419; 548/511

(57) ABSTRACT

The invention relates to compounds of the general formula (I) 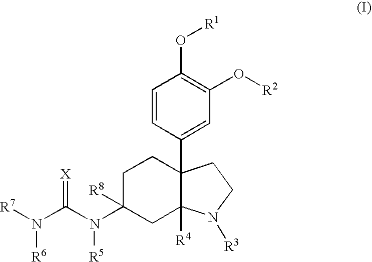
wherein R¹, R², R³, R⁴, R⁵, R⁶, R⁷, R⁸, and X are as defined in the description, or pharmaceutically acceptable salts, hydrates, geometrical isomers, racemates, tautomers, optical isomers, N-oxides and prodrug forms thereof. The compounds may be used for the treatment or prophylaxis of disorders related to the MCH1R receptor and for modulation of appetite. The invention also relates to such use as well as to pharmaceutical formulations comprising a compound of formula (I).

### RELATED APPLICATIONS

This application claims priority to Swedish application number 0303181-2, filed on Nov. 26, 2003, and U.S. provisional application 60/549,644, filed on Mar. 3, 2004, the contents of which is incorporated herein by reference.

### TECHNICAL FIELD

The present invention relates to substituted octahydroindoles that act as antagonist for the melanin concentrating hormone receptor 1 (MCH1R). The invention further relates to pharmaceutical compositions comprising these compounds, the use of the compounds for the preparation of a medicament for the prophylaxis and treatment of obesity, and methods for the prophylaxis and treatment of obesity.

### BACKGROUND

Melanin Concentrating Hormone (MCH) is a 19 AA cyclic peptide, which is expressed in hypothalamus in the mammalian brain (Nahon J L et al., Endocrinology, 1989; 125(4):2056-65 and Tritos N A, et al., Diabetes, 1998; 47(11):1687-92). A substantial body of evidence has shown that this peptide plays a critical role in the moderation of feeding behavior and energy expenditure. Studies have shown that ICV administration of MCH directly into rat brains results in a marked increase in food intake (Ludwig D S et al., Am. J. Physiol., 1998; 274(4 Pt 1):E627-33). It has also been shown that messenger RNA for the MCH precursor is up-regulated in the hypothalamus of fasted animals and in animals that are genetically obese (Qu D, Ludwig D S et al., Nature, 1996; 380(6571):243-7). Furthermore, mice lacking MCH are hypophagic and lean, and have increased energy expenditure (20% increase over control animals when expressed on a per kg basis) (Shimada M et al., Nature, 1998; 396(6712):670-4). Studies of transgenic mice overexpressing MCH in the lateral hypothalamus show that these animals are more prone to diet-induced obesity when fed a high fat diet, and they have higher systemic leptin levels (Ludwig D S et al., J. Clin. Invest., 2001; 107(3):379-86). Blood glucose levels were increased both preprandially and after intraperitoneal injection of glucose. The animals also had increased insulin levels and insulin tolerance test indicated peripheral insulin resistance. Further support for the role of MCH in metabolic regulation comes from studies showing that, in mice, mRNA for the MCH receptor is upregulated 7-fold by 48 h fasting and in genetic leptin deficiency (ob/ob mice). These effects could be completely blunted by leptin treatment (Kokkotou E G et al., Endocrinology, 2001; 142(2):680-6.). In addition to its role in regulating feeding behavior, MCH antagonists have been demonstrated to have anxiolytic and antidepressant effects (Borowsky, B D et al., Nature Medicine, 2002. 8(8): 825-830).

Obesity is linked to a wide range of medical complications, such as diabetes, cardiovascular disease and cancer. In addition, being overweight can exacerbate the development of osteoporosis and asthma. For example, at least 75% of Type II diabetics are overweight and a clear correlation has been demonstrated between weight and the prevalence of Type II diabetes. Obesity is also proven to double the risk of hypertension. It is estimated that between 2% and 8% of total health-care costs in the Western world are related to obesity, i.e., in excess of 10 billion USD.

Initial treatment for obesity is simple diet and exercise. Initial drug therapy tends to be focused around suppression of appetite. Many of the older appetite-suppressant agents act via the noradrenergic (and possibly dopaminergic) receptors to produce a feeling of satiety. Amphetamine was the archetypal agent in this class, but it has substantial potential for stimulating the central nervous system and consequent abuse. More recent developments, such as Xenical® (orlistat), marketed by Roche, have focused on preventing fat absorption in the gut. Xenical® inhibits the action of the enzyme lipases, thereby reducing the digestion of triglycerides and subsequent absorption by the intestinal tract. Unfortunately, this does not address overeating and excess calorie intake. Other pharmacological approaches for the treatment of obesity include serotonin re-uptake inhibitors, such as Reductil® (sibutramine) marketed by Abbot, which acts as an appetite-suppressant.

The concept of using MCH1R antagonists for the treatment of obesity has recently been published. A review is presented by Carpenter and Hertzog, Expert Opin. Ther. Patents, 2002, 12(11): 1639-1646.

WO01/21169 (Takeda Chemical Industries) describes diaryl compounds as MCH-1R antagonists useful for the treatment of obesity. Also JP13226269 (Takeda), describing several piperidine-substituted benzazepines and benzazepinones; WO01/82925 (Takeda), disclosing different amines; and WO01/87834 (Takeda) describing piperidine compound with benzene (1:1), claim compounds for the treatment of obesity. WO01/21577 (Takeda) discloses a series of amines claimed to be anorectic, antidiabetic and antidepressant agents.

WO01/57070 (Merck) describes in a series of truncated and modified peptidic MCH analogues as either significant agonist or antagonist activity. In WO02/10146 (GlaxoSmithKline) the preparation of carboxamide compounds claimed for the treatment of obesity as well as diabetes, depression and anxiety is disclosed. WO02/04433 (The Neurogen Corporation) describes in N-arylpiperazine derivatives and related 4-arylpiperidine derivatives as selective modulators of MCH-1R for the treatment of a variety of metabolic, feeding and sexual disorders. In WO02/06245 (Synaptic Pharmaceutical Corporation) a class of dihydropyrimidinones as MCH-1R antagonists for the treatment of feeding disorders, such as obesity and bulimia is disclosed. In WO02/051809 (Schering Corporation) 4-substituted piperidine derivatives are disclosed as MCH antagonists as well as their use in the treatment of obesity. In WO02/057233 aryl-substituted ureas are disclosed as MCH antagonists as well as their use in the treatment of obesity. The central core in the WO02/057233 is an arylene or heteroarylene group, whereas the central core in the present compounds is an octahydroindole group.

Mesembrine, 3a-(3,4-dimethoxyphenyl)-1-methyloctahydro-6H-indol-6-one, is a natural product obtained as an extract of plants of the Mesembryanthemaceae family, including Sceletium tortuosum. In small doses the mesembrine have a meditative or narcotic effect. Hottentots used Sceletium expansum and tortuosum as a psychedelic called “channa”. The use of mesembrine as a serotonin-uptake inhibitor for the treatment of an array of mental disorders is disclosed in WO97/46234.

Parkes et al., Journal of Neuroendocrinology 1996, 8, 57-63 has shown that the MCH-peptide acts as a diuretic. Therefore, MCH antagonists should work as anti-diuretics.

U.S. Pat. No. 6,288,104 discloses mesembrine-like compounds lacking the urea group in the present compounds. This document relates to serotonin-uptake inhibitors for the treatment of depression and anxiety, whereas the present compounds are antagonists for the MCH-1R.

None of the above disclosures discloses the compounds according to the present invention as antagonists for the MCH-1R.

### SUMMARY

According to the present invention, novel substituted octahydroindoles have been found that are active towards the MCH1R receptor. The compounds are relatively easy to prepare and can be used for the treatment or prevention of obesity, diabetes mellitus, hyperlipidemia, hyperglycemia, modulation of appetite, depression, anxiety or urinary incontinence. The compounds can further be used in conjunction with other compounds acting through other mechanisms, such as MC-4 agonists, 5HT_{2c} agonists, or 5HT₆ antagonists. The compounds can also be used in conjunction with anti-obesity medicaments.

Definitions

The following definitions shall apply throughout the specification and the appended claims.

Unless otherwise stated or indicated, the term “C₁₋₆-alkyl” denotes a straight or branched alkyl group having from 1 to 6 carbon atoms. Examples of said lower alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. “Halo-C₁₋₆-alkyl” means a C₁₋₆-alkyl group substituted by one or more halogen atoms.

Unless otherwise stated or indicated, the term “C₃₋₈-cycloalkyl” denotes a cyclic alkyl group having a ring size from 3 to 8 carbon atoms. Examples of said cycloalkyl include cyclopropyl, cyclobutyl, cyclopentyl, cyclohexyl, methylcyclohexyl, cycloheptyl, and cyclooctyl. For parts of the range “C₃₋₈-cycloalkyl” all subgroups thereof are contemplated such as C₃₋₇-cycloalkyl, C₃₋₆-cycloalkyl, C₃₋₅-cycloalkyl, C₃₋₄-cycloalkyl, C₄₋₈-cycloalkyl, C₄₋₇-cycloalkyl, C₄₋₆-cycloalkyl, C₄₋₅-cycloalkyl, C₅s₇-cycloalkyl, C₆₋₇-cycloalkyl, etc.

Unless otherwise stated or indicated, the term “C₁₋₆ alkoxy” denotes a straight or branched alkoxy group having from 1 to 6 carbon atoms. Examples of said lower alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc. “Halo-C₁₋₄-alkoxy” means a C₁₋₆-alkoxy group substituted by one or more halogen atoms.

Unless otherwise stated or indicated, the term “C₂₋₄-alkenyl” denotes a straight or branched alkenyl group having from 2 to 6 carbon atoms. Examples of said alkenyl include vinyl, allyl, 1-butenyl, 1-pentenyl, and 1-hexenyl. For parts of the range “C₂₋₆-alkenyl” all subgroups thereof are contemplated such as C₂₋₅-alkenyl, C₂₋₄-alkenyl, C₂₋₃-alkenyl, C₃₋₄-alkenyl, C₃₋₅-alkenyl, C₃₋₄-alkenyl, C₄₋₆-alkenyl, C₄₋₅-alkenyl, etc.

Unless otherwise stated or indicated, the term “halogen” shall mean fluorine, chlorine, bromine or iodine.

Unless otherwise stated or indicated, the term “aryl” refers to a hydrocarbon ring system having at least one aromatic ring. Examples of aryls are phenyl, pentalenyl, indenyl, isoindolinyl, chromanyl, naphthyl, fluorenyl, anthryl, phenanthryl and pyrenyl. The aryl rings may optionally be substituted by C₁₋₆-alkyl. Examples of substituted aryl groups are benzyl and 2-methylphenyl.

The term “heteroaryl” refers to a hydrocarbon ring system having at least one aromatic ring which contains at least one heteroatom such as O, N, or S. Examples of heteroaryl groups include furyl, pyrrolyl, thienyl, oxazolyl, imidazolyl, thiazolyl, pyridinyl, pyrimidinyl, quinazolinyl, and indolyl groups.

The term “heterocyclo” refers to a hydrocarbon ring system containing 4 to 8 ring members that have at least one heteroatom (e.g., S, N, or O) as part of the ring. It includes saturated, unsaturated, aromatic, and nonaromatic heterocycles. Suitable heterocyclo groups include thienyl, furyl, pyridyl, pyrrolidinyl, imidazolyl, pyrazolyl, piperidyl, azepinyl, morpholinyl, pyranyl, dioxanyl, pyridazinyl, pyrimidinyl, and piperazinyl groups.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8^{th} ed., Mc-Graw-Hill, Int. Ed. 1992, “Biotransformation of Drugs”, p. 13-15.

When two of the above-mentioned terms are used together, it is intended that the latter group is substituted by the former. For example, aryl-C₁₋₆ alkyl means a C₁₋₆-alkyl group that is substituted by an aryl group. Likewise, halo C₁₋₄ alkoxy means a C₁₋₆-alkoxy group that is substituted by one or more halogen atoms.

The following abbreviations have been used:
(Boc)₂O means di-tert-butyl carbonate,
DCM means dichloromethane,
DIBAL-H means diisobutylaluminium hydride,
DMF means dimethylformamide,
HPLC means high performance liquid chromatography,
Hunig's base means N-ethyldiisopropylamine,
PNP means para-nitrophenyl,
R.T. (rt) means room temperature,
TFA means trifluoroacetic acid,
THF means tetrahydrofuran.

In a first aspect, the present invention provides a compound of the general formula (I) 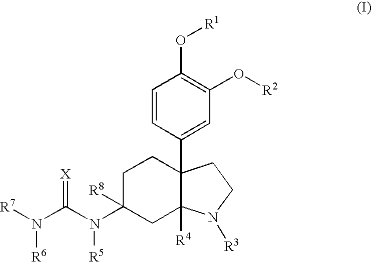
or a pharmaceutically acceptable salt, hydrates, geometrical isomers, racemates, tautomers, optical isomers, N-oxides and prodrug forms thereof, wherein:
R¹ is C₁₋₆-alkyl;
R² is C₁₋₆-alkyl;
or R¹ and R² are linked to form C₁₋₃-alkylene;
R³ is H, C₁₋₆-alkyl, C₁₋₆-alkoxycarbonyl, C₁₋₆-alkoxycarbonyl-C₁₋₆-alkyl, C₁₋₆-alkoxycarbonyl-C₃₋₆-cycloalkyl-C₁₋₆ alkyl, aryl-C₁₋₆-alkyl, heteroaryl-C₁₋₆ alkyl, aryl or heteroaryl, wherein any aryl or heteroaryl may be unsubstituted or independently substituted with C₁₋₆-alkyl, C₁₋₆-alkoxy, C₁₋₆-alkoxycarbonyl, C₁₋₆-alkoxycarbonyl-C₁₋₆-alkyl, C₁₋₆-alkoxycarbonyl-C₃₋₆-cycloalkyl-C₁₋₆ alkyl, halo-C₁₋₆-alkyl, halo-C₁₋₆-alkoxy, or nitro;
R⁴ is H or C₁₋₆-alkyl;
R⁵ is H, OH, C₁₋₆-alkyl, C₁₋₆-alkylaminocarbonyl, or C₁₋₆-alkylaminothioxylcarbonyl;
R⁶ is H or C₁₋₆-alkyl;
R⁷ is H, C₁₋₆-alkyl, C₂₋₆-alkenyl, C₃₋₈-cycloalkyl, C₃₋₈-cycloalkyl-C₁₋₆-alkyl, C₁₋₆-alkoxy, C₁₋₆-alkoxy-C₁₋₆-alkyl, C₁₋₆-alkylcarbonyl, C₁₋₆-alkoxycarbonyl, C₁₋₆-alkoxycarbonyl-C₁₋₆-alkyl, heterocyclo-C₁₋₆-alkyl, heteroaryl-C₁₋₆-alkyl, aryl-C₁₋₆-alkyl, arylcarbonyl, heteroarylcarbonyl, or heterocyclyl, wherein any aryl, heteroaryl and heterocyclyl may be unsubstituted or substituted in one, two or three positions with C₁₋₆-alkyl, halo-C₁₋₆-alkyl, C₁₋₆-alkoxy, or halogen;
or R⁶ and R⁷ are linked to form C₄₋₆-alkylene or together with the nitrogen atom to which they are attached form a piperazine ring, which may be unsubstituted or substituted in one position with C₁₋₆-alkyl;
R⁸ is H or C₁₋₆-alkyl; and
X is O, S, NH, CH-NO₂, or NCN.

In some embodiments R¹ and R² are methyl.

In some embodiments that R¹ and R² are linked to form methylene.

In some embodiments R⁴ is H.

In some embodiments R⁸ is H.

In some embodiments when X is S:
R³ is hydrogen; methyl; ethyl; n-propyl; isobutyl; pentyl; tert-butoxycarbonyl; ethoxycarbonylmethyl; ethoxycarbonylcyclopropylmethyl; benzyl, which may be unsubstituted or independently substituted in one or two positions with trifluoromethyl, methoxy, trifluoromethoxy, and nitro; phenylethyl; phenylpropyl; furylmethyl; methyl-furylmethyl; thienylmethyl; methyl-pyrrolylmethyl; pyridylmethyl; methyl-1H-imidazolylmethyl; or quinolinylmethyl;
R⁵ is H or methyl;
R⁶ is H; and
R⁷ is ethyl, n-butyl, isobutyl, sec-butyl, tert-butyl, hexyl, cyclopropyl, cyclopentyl, cyclopropylmethyl, allyl, methoxypropyl, morpholinylethyl, furylmethyl, benzyl, phenylethyl, fluorobenzyl, or methoxybenzyl.

In some embodiments when X is O:
R³ is methyl or benzyl;
R⁵ is H;
R⁶ is H or methyl;
R⁷ is ethyl, allyl, benzyl, ethoxycarbonylethyl, piperazinylethyl, piperazinylpropyl, methylbenzyl, fluorobenzyl, bromobenzyl, dichlorobenzyl, methoxybenzyl, trifluoromethylbenzyl, phenylethyl, thienylethyl, pyridinylmethyl, methylpyrazinylmethyl, or 4-methylpiperazin-1-yl; or that
R⁶ and R⁷ together with the nitrogen to which they are attached form a piperazine, which may be unsubstituted or substituted in one position with methyl.

In some embodiments hen X is NH:
R³ is methyl;
R⁵ and R⁶ both are H; and
R⁷ is n-butyl, n-pentyl, or benzyl.

Certain specific embodiments are denoted in Examples 10-76 and 78-95 below.

All diastereomeric forms possible (pure enantiomers, tautomers, racemic mixtures and unequal mixtures of two enantiomers) are within the scope of the invention. Such compounds can also occur as cis- or trans-, E- or Z-double bond isomer forms. All isomeric forms are contemplated.

Another object of the present invention is a process for the preparation of a compound above comprising at least one of the following steps:
(a) coversion of a nitrile to a cyclopropanecarbonitrile,
(b) reduction of the cyclopropanecarbonitrile to give a cyclopropanecarbaldehyde,
(c) reaction of the cyclopropanecarbaldehyde to give an imine and further reaction of the imine with α,β-unsaturated ketone to form an octahydro-indol-6-ketone,
(d) treatment of the octahydro-indol-6-ketone with ammonia, a first amine or a salt thereof and then reduction to give a second amine,
(e) reaction of the second amine with an isocyanate, isothiocyanate, triphosgene, carboximidamide, or chloroformate.

Another object of the present invention is a compound as described above for use in therapy. The compound can be used in the treatment or prophylaxis of obesity, diabetes mellitus, hyperlipidemia, hyperglycemia, depression, anxiety, urinary incontinence, and for modulation of appetite. It may also be used in the treatment or prophylaxis of disorders relating to the MCH1R receptor and for modulation of appetite. Examples of such disorders are obesity, diabetes mellitus, hyperlipidemia, hyperglycemia, depression, anxiety, and urinary incontinence. The compound can further be used in conjunction with other compounds active towards other receptors, such as MC-4 agonists, 5HT_{2c}, agonists, or 5HT₆ antagonists. The compound can also be used in conjunction with anti-obesity medicaments.

Another object of the present invention is a pharmaceutical formulation containing a compound as described above as an active ingredient, in combination with a pharmaceutically acceptable diluent or carrier. The pharmaceutical formulation may be used in the treatment or prophylaxis of obesity wherein the active ingredient is a compound as described above.

Another object of the present invention is a method for the treatment or prophylaxis of obesity, diabetes mellitus, hyperlipidemia, hyperglycemia, depression, anxiety, urinary incontinence, and for modulation of appetite, said method comprising administering to a subject (e.g., mammal, human, or animal) in need of such treatment (e.g., including the step of identifying the subject as in need of such treatment) an effective amount of a compound as described above. The compound can further be used in conjunction with other compounds active towards other receptors, such as MC-4 agonists, 5HT_{2c} agonists, or 5HT₆ antagonists. The compound can also be used in conjunction with anti-obesity medicaments.

Another object of the present invention is a method for the treatment or prophylaxis of disorders related to the MCH1R receptor and for modulation of appetite, said method comprising administering to a subject (e.g., mammal, human, or animal) in need of such treatment an effective amount of a compound as described above. The MCH1R receptor related disorder is any disorder or symptom wherein the MCH1R receptor is involved in the process or presentation of the disorder or the symptom. The MCH1R related disorders include, but are not limited to obesity, diabetes mellitus, hyperlipidemia, hyperglycemia, depression, anxiety, and urinary incontinence. The compound can further be used in conjunction with other compounds active towards other receptors, such as MC-4 agonists, 5HT₂c agonists, or 5HT₆ antagonists. The compound can also be used in conjunction with anti-obesity medicaments.

The methods delineated herein can also include the step of identifying that the subject is in need of treatment of the MCH1R receptor-related disorder. Identifying a subject in need of such treatment can be in the judgment of a subject or a health care professional and can be subjective (e.g., opinion) or objective (e.g., measurable by a test or diagnostic method).

Another object of the present invention is a method for modulating MCH1R receptor activity (e.g., antagonizing the human MCH1R receptor), comprising administering to a subject (e.g., mammal, human, or animal) in need thereof an effective amount of a compound as described above or a composition comprising a compound as described above.

Another object of the present invention is the use of a compound as described above in the manufacture of a medicament for use in the treatment or prophylaxis of obesity, diabetes mellitus, hyperlipidemia, hyperglycemia, depression, anxiety, and urinary incontinence, and for modulation of appetite.

Another object of the present invention is the use of a compound as described above in the manufacture of a medicament for use in the treatment or prophylaxis of disorders related to the MCH1R receptor and for modulation of appetite, said method comprising administering to a subject (e.g., mammal, human, or animal) in need of such treatment an effective amount of a compound as described above. The MCH1R receptor related disorder is any disorder or symptom wherein the MCH1R receptor is involved in the process or presentation of the disorder or the symptom. The MCH1R related disorders include, but are not limited to obesity, diabetes mellitus, hyperlipidemia, hyperglycemia, depression, anxiety, and urinary incontinence. The compound can further be used in conjunction with other compounds active towards other receptors, such as MC-4 agonists, 5HT_{2c} agonists, or 5HT₆ antagonists. The compound can also be used in conjunction with anti-obesity medicaments.

### DETAILED DESCRIPTION

The compounds of the formula (I) may be used as such or, where appropriate, as pharmacologically acceptable salts (acid or base addition salts) thereof. The pharmacologically acceptable addition salts mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulfuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulfonic acid, toluenesulfonic acid, methanesulfonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

In a further aspect the invention relates to methods of making compounds of any of the formulae herein comprising reacting any one or more of the compounds of the formulae delineated herein, including any processes delineated herein. The compounds of the formula (I) above may be prepared by, or in analogy with, conventional methods.

The processes described above may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g. as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The necessary starting materials for preparing the compounds of formula (I) are either known or may be prepared in analogy with the preparation of known compounds. The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The invention will now be further illustrated by the following specific examples. These examples are not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

General Comments

¹H nuclear magnetic resonance (NMR) and ¹³C NMR were recorded on a Bruker PMR 500 spectrometer at 500.1 MHz and 125.1 MHz, respectively or on a JEOL eclipse 270 spectrometer at 270.0 MHz and 67.5 MHz, respectively, or on a Bruker Advance DPX 400 spectrometer at 400.1 and 100.6 MHz, respectively. All spectra were recorded using residual solvent or tetramethylsilane (TMS) as internal standard. All spectra were recorded using residual solvent or tetramethylsilane (TMS) as internal standard. IR spectra were recorded on a Perkin-Elmer Spectrum 1000 FT-IR spectrometer. Electrospray mass spectrometry (MS) were obtained using an Agilent MSD mass spectrometer. Accurate mass measurements were performed on a Micromass LCT dual probe. Elemental analyses were performed on a Vario El instrument or sent to Mikro Kemi in Uppsala. Analytical HPLC were performed on Agilent 1100. Preparative HPLC was performed on a Gilson system or on a Waters/Micromass Platform ZQ system. Preparative flash chromatography was performed on Merck silica gel 60 (230-400 mesh). The compounds were automatically named using ACD6.0. GC-MS analysis was performed on a Hewlett Packard 5890 gas chromatograph with a HP-5MS 15 m*0.25 mm*0.25 μm column connected to a 5971 MS detector. Electrospray mass spectrometry (MS) spectra were obtained on a Perkin-Elmer API 150EX mass spectrometer. Accurate mass measurements were performed on a Micromass LCT dual probe.
General Procedures for the Preparation of Compounds of the Present Invention

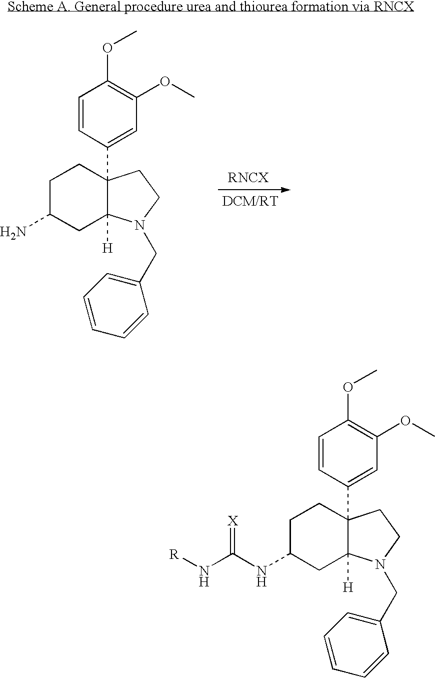

A solution of the amine, (3aS*,6R*,7aS*)-1-benzyl-3a-(3,4-dimethoxyphenyl)octahydro-1H-indol-6-amine, Comparative Example 5 (18.3 mg; 0.05 mmol) in methylene chloride (2.0 ml) was treated with an isocyanate or isothiocyanate (1 equiv.; 0.05 mmol) The mixture was shaken at room temperature for 18 h, then the solvent was removed by evaporation.

The residues were purified by preparative HPLC.

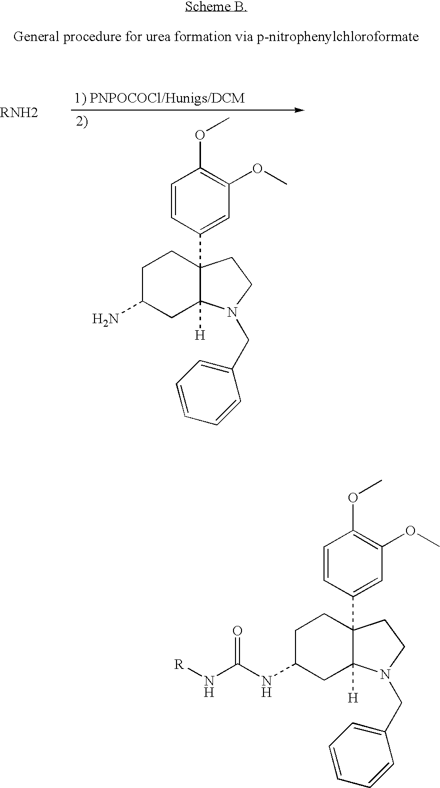

A solution of the appropriate amine (1 mmol) in DCM (5.0 ml) was treated with para-nitrophenylchloroformate (1 mmol). The resulting solution was then treated dropwise at room temperature with Hunigs base (1 mmol). The mixtures were stirred at room temperature for 5 h.

An aliquot (0.25 ml; 0.05 mmol) of the crude PNP-carbamate from the reaction mixtures described above was then transferred to a solution of the amine, (3aS*,6R*,7aS*)-1-benzyl-3a-(3,4-dimethoxyphenyl)octahydro-1H-indol-6-amine, Comparative Example 5 (18 mg; 0.05 mmol) in methylene chloride (3.0 ml) and the resulting solution shaken at R.T. overnight.

The solvent was removed by evaporation and the crude reaction mixtures purified by preparative HPLC.

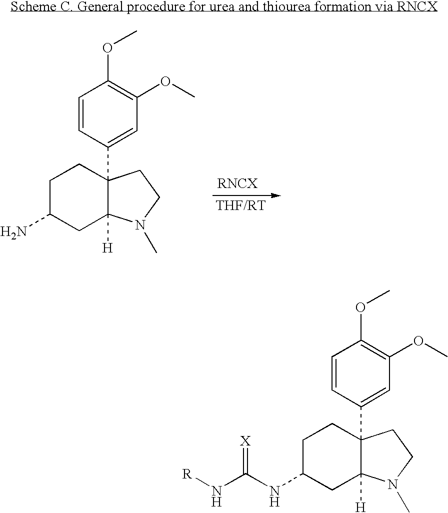

A solution of the amine, (3aS*,6R*,7aS*)-1-methyl-3a-(3,4-dimethoxyphenyl)octahydro-1H-indol-6-amine, Comparative Example 7 (7.3 mg; 0.025 mmol) in tetrahydrofuran (1.0 ml) was treated with and isocyanate or isothiocyanate (1 equiv.; 0.025 mmol) The mixture was shaken at room temperature for 18 h, then the solvent was removed by evaporation.

The residues were purified by preparative HPLC.

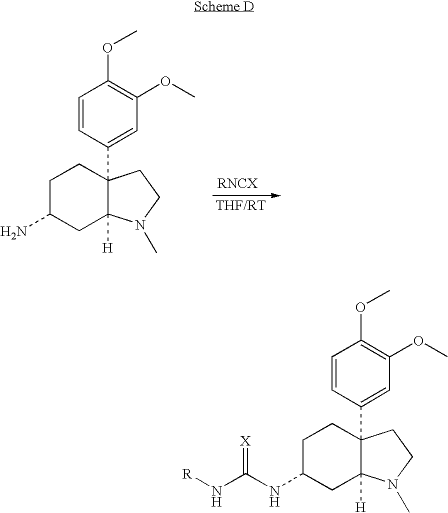

The amine, (3aS*,6R*,7aS*)-1-methyl-3a-(3,4-dimethoxyphenyl)octahydro-1H-indol-6-amine, Comparative Example 7 (7 mg, 0.024 mmol) and isocyanate (1.3 eq) were dissolved in dry THF (1.5 ml). Reaction in R.T., under N₂ and overnight. The solvent was evaporated under reduced pressure. Purification was performed by preparative HPLC.

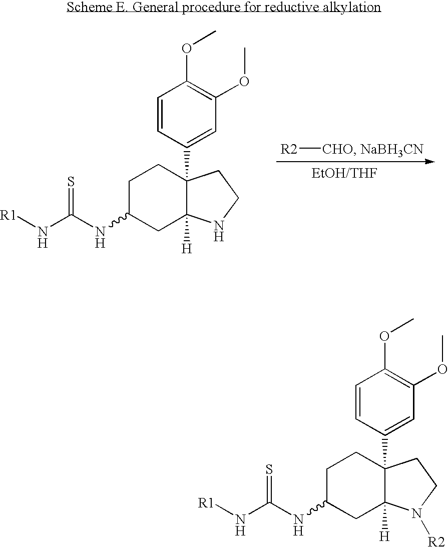

To the appropiate amine (0.06 mmol) in EtOH (0.5 ml) and THF (0.5 ml) was added an aldehyde (0.1 mmol) and NaBH₃CN (1 mmol). The mixture was stirred overnight and concentrated. 2 M NaOH was added and the aqueous layer extracted with EtOAc. The products were purified by preparative HPLC.

Synthesis of Starting Materials (Examples 1-9)

### COMPARATIVE EXAMPLE 1

### 1-(3,4-dimethoxyphenyl)cyclopropanecarbonitrile

Dimethoxyphenyl acetonitrile (4.43 g, 2.5 mmol) was dissolved in DMF (20 mL). Sodium hydride (4 g of a 60% dispersion, 2.4 g, 100 mmol) was added in portions and the mixture was stirred at room temperature for 10 minutes. Bromochloroethane (2.1 mL, 3.62 g, 25.2 mmol) was added, and the mixture stirred at room temperature overnight. The reaction was cautiously quenched by addition of a methanol/water mixture (1:1, 300 mL) and the reaction products were extracted into ethyl acetate (3×200 mL). The combined extracts were washed with water (4×200 mL), brine (1×200 mL) and then dried (Na₂SO₄). The solvent was then removed under reduced pressure and the crude product chromatographed (SiO₂, EtOAc/petroleum ether 1:3 as eluent) to give the title compound as an off-white solid (2.4 g, 47%). ¹H NMR (270 MHz, CDCl₃) δ 1.32 (m, 2H) 1.64 (m, 2H) 3.84 (s, 3H) 3.88 (s 3H) 6.79 (d, J=1.0 Hz, 2H) 6.84 (s 1H). MS (ESI+) for C₁₂H₁₃NO₂: m/z 204.1 (M+1).

### COMPARATIVE EXAMPLE 2

### 1-(3,4-dimethoxyphenyl)cyclopropanecarbaldehyde

1-(3,4-dimethoxyphenyl)cyclopropanecarbonitrile, Comparative Example 1 (2.0 g, 9.84 mmol) was dissolved in THF (30 mL). DIBAL-H (15 mL of a 1.0 M solution in toluene, 15 mmol) was added and the mixture was stirred at room temperature for 3 hours. The reaction was cautiously quenched by addition of 2 M HCl and organic components were extracted into dichloromethane (3×125 mL). The combined extracts were washed with water (2×100 mL), brine (2×100 mL) and then dried (Na₂SO₄), giving the title compound as an off-white sold (1.95 g, 98%). ¹H NMR (270 MHz, CDCl₃) δ ppm 1.38 (m, 2H) 1.53 (m, 2H) 3.87 (s, 6H) 6.81 (s, 1H) 6.84 (d, J=1.0 Hz, 2H) 9.23 (s, 1H). MS (ESI+) for C₁₂H₁₄O₃: no ion detected.

### COMPARATIVE EXAMPLE 3

### (3aS*,7aS*)-1-benzyl-3a-(3,4-dimethoxyphenyl)octahydro-6H-indol-6-one

1-(3,4-dimethoxyphenyl)cyclopropanecarbaldehyde, Comparative Example 2 (3.25 g, 16.0 mmol) was dissolved in dichloromethane (35 mL). Benzylamine (1.77 mL, 1.74 g, 16.2 mmol) was added, followed by sodium sulfate (15 g, 105.6 mmol). The mixture was stirred at room temperature overnight before being filtered and evaporated to yield the crude imine as a clear oil. This material was then dissolved in DMF (15 mL), and sodium iodide (246 mg, 1.64 mmol) and trimethylsilyl chloride (202 μL, 172 mg, 1.58 mmol) were added. The resulting mixture was heated to 70° C. for 3 hours and then partitioned between water (150 mL) and ethyl acetate (200 mL). The aqueous phase was extracted with a further portion of ethyl acetate (1×200 mL) and the combined extracts were washed with brine (1×200 mL) and dried (Na₂SO₄). The solvent was removed under reduced pressure, and the crude product dissolved in dichloromethane (30 mL). To this was added HCl in ether (70 mL of a 1.0 M solution, 70 mmol) and the crude HCl salt was evaporated to dryness. This material was then dissolved in acetonitrile (70 mL), methyl vinyl ketone (1.42 mL, 1.19 g, 17 mmol) was added and the mixture heated to reflux for 16 hours. On cooling the solvent was removed under reduced pressure and the resulting dark oil partitioned between 3M HCl solution (200 mL) and ether (150 mL). The aqueous fraction was washed with further ether (3×150 mL), and then brought to basic pH using 3 M NaOH solution. The organic components were then extracted into diethyl ether (3×150 mL) and the combined extracts washed with brine (1×200 mL) and dried (Na₂SO₄). On removal of the solvent under reduced pressure, the crude product was purified by chromatography (SiO₂, ethyl acetate/petroleum ether 2:3 as eluent) to give the title compound as a clear oil (3.10 g, 53%).

¹H NMR (270 MHz, CDCl₃) δ ppm: 1.87-2.38 (m, 6H); (2.38-2.82 (m, 3H); 2.82-3.05 (m, 1H); 3.05-3.20 (m, J=12.6 Hz, 1H); 3.20-3.35 (m, 1H); 3.92 (s, 6H); 3.96-4.19 (m, J=12.6 Hz, 1H); 6.73-7.03 (m, 3H) 7.09-7.42. ¹³C NMR (68 MHz, CDCl₃) δ ppm: 34.80, 36.21, 38.61, 40.63, 47.18, 51.67, 53.38, 57.38, 60.32, 68.15, 109.90, 110.95, 117.76, 126.89, 128.15, 128.76, 138.79, 140.32, 147.47, 148.98, 211.36.

### COMPARATIVE EXAMPLE 4

### (3aS*,7aS*)-3a-(3,4-dimethoxyphenyl)-1-methyloctahydro-6H-indol-6-one

1-(3,4-dimethoxyphenyl)cyclopropanecarbaldehyde, Comparative Example 2 (8.0 g, 38.8 mmol) was dissolved in dichloroethane (100 mL). Sodium sulfate (25 g, 176 mmol) was added and methylamine gas was bubbled through the solution for 10 minutes. The reaction vessel was then sealed and the mixture stirred at room temperature overnight before being filtered and evaporated to yield the crude imine as a yellow oil. This material was then dissolved in DMF (30 mL), and sodium iodide (585 mg, 3.90 mmol) and trimethylsilyl chloride (500 L, 426 mg, 3.92 mmol) were added. The resulting mixture was heated to 90° C. for 3 hours and then partitioned between water (200 mL) and ethyl acetate (200 mL). The aqueous phase was extracted with a further ethyl acetate (2×100 mL) and the combined extracts were dried (Na₂SO₄). The solvent was removed under reduced pressure, and the crude product dissolved in dichloromethane (100 mL). To this was added HCl in ether (100 mL of a 1.0 M solution, 100 mmol) and the crude HCl salt was evaporated to dryness. This material was then dissolved in acetonitrile (100 mL), methyl vinyl ketone (3.5 mL, 2.95 g, 42.1 mmol) was added and the mixture heated to reflux for 16 hours. On cooling the solvent was removed under reduced pressure and the resulting dark oil partitioned between 3M HCl solution (200 mL) and ether (200 mL). The aqueous fraction was washed with further ether (2×100 mL), and then brought to basic pH using 3 M NaOH solution. The organic components were then extracted into ethyl acetate (4×150 mL) and the combined extracts washed with brine (1×200 mL) and dried (Na₂SO₄). On removal of the solvent under reduced pressure, the crude product was purified by chromatography (SiO₂, ethyl acetate as eluent) to give the title compound as a yellow oil (4.5 g, 40%).

¹H NMR (270 MHz, CDCl₃) δ ppm: 1.99-2.12 (m, 2H); 2.12-2.26 (m, 3H); 2.28 (s, 3H); 2.30-2.47 (m, 2H); 2.52-2.62 (m, 2H); 2.88-2.95 (m, 1H) 3.06-3.15 (m, 1H) 3.85 (s, 3H); 3.87 (s, 3H); 6.76-6.93 (m, 3H). ¹³C NMR (68 MHz, CDCl₃) δ ppm: 35.20, 36.16, 38.76, 40.01, 40.48, 47.42, 54.78, 55.82, 55.92, 70.31, 109.84, 110.87, 117.83, 140.12, 147.39, 148.90, 211.40. MS (ESI+) for C₁₇H₂₃NO₃ m/z 290.2 (M+H)⁺. HRMS (EI) calcd for C₁₇H₂₃NO₃: 289.1678, found 289.1684

### COMPARATIVE EXAMPLE 5

### (3aS*,6R*,7aS*)-1-benzyl-3a-(3,4-dimethoxyphenyl)octahydro-1H-indol-6-amine and

### COMPARATIVE EXAMPLE 6

### (3aS*,6S*,7aS*)-1-benzyl-3a-(3,4-dimethoxyphenyl)octahydro-1H-indol-6-amine

(3 aS*,7aS*)-1-benzyl-3a-(3,4-dimethoxyphenyl)octahydro-6H-indol-6-one (Comparative Example 3) (750 mg, 2.05 mmol) was dissolved in methanol (60 mL). Ammonium acetate (1.6 g, 20.8 mmol) was added and the solution allowed to stir at room temperature for 2 hours before sodium cyanoborohydride (100 mg, 1.59 mmol) was added. The mixture was stirred at room temperature for 16 hours, diluted with 3 M NaOH solution (100 mL) and extracted into dichloromethane (2×150 mL). The combined extracts were dried (Na₂SO₄) and the solvent removed to give the crude mixture of amines (410 mg, 55%). This crude material was used as a mixture without further purification, or the cis (6R*)- and trans-isomer (6S*) separated by flash chromatography using chloroform saturated with NH₃ (g).

### COMPARATIVE EXAMPLE 7

### (3aS*,6R*,7aS*)-1-methyl-3a-(3,4-dimethoxyphenyl)octahydro-1H-indol-6-amine and

### COMPARATIVE EXAMPLE 8

### (3aS*,6S*,7aS*)-1-methyl-3a-(3,4-dimethoxyphenyl)octahydro-1H-indol-6-amine

Same procedure as for Comparative Example 5 and Comparative Example 6 starting from (3aS*,7aS*)-3a-(3,4-dimethoxyphenyl)-1-methyloctahydro-6H-indol-6-one (Comparative Example 4). This crude material was used as a mixture without further purification, or the cis (6R*)- and trans-isomer (6S*,) separated by flash chromatography using chloroform saturated with NH₃ (g).

### COMPARATIVE EXAMPLE 9

### tert-butyl (3aS*,7aS*)-3a-(3,4-dimethoxyphenyl)-6-oxooctahydro-1H-indole-1-carboxylate

Into a solution of Comparative Example 3 (3.0 g, 8.2 mmol) and (Boc)₂O (3.0 g, 13.7 mmol) in i-PrOH (200 mL) was suspended 10% Pd on charcoal (0.8 g), and the resulting mixture was vigorously agitated under H₂ (1.4 atm) during 4 h at rt. The catalyst was filtered off and the filtrate was shaken with PS-trisamine (3.0 g, 4 mmol/g) at rt overnight. The resin was filtered off and the solvent evaporated, leaving the title compound (2.4 g, 80%) as a thick oil, which was used in the next step without further purification.

¹H NMR (270 MHz, CDCl₃): δ ppm 1.28-1.51 (m, 9H), 1.96-2.38 (m, 6H), 2.43-2.72 (m, 1H), 2.72-2.89 (m, 1H), 3.14-3.45 (m, 1H), 3.68-3.84 (m, 6H), 4.27-4.58 (m, 1H), 6.60-6.84 (m, 3H).

¹³C NMR (270 MHz, CDCl₃): δ ppm 14.22, 21.04, 28.50, 33.22, 36.59, 44.76, 55.90, 55.96, 60.29, 79.87, 100.00, 109.49, 111.20, 117.99, 137.66, 147.86, 149.08, 210.41.

### SYNTHESIS OF EXAMPLES 10-95

### EXAMPLE 10

### N-[(3aS*,6S*,7aS*)-1-benzyl-3a-(3,4-dimethoxyphenyl)octahydro-1H-indol-6-yl]-N′-ethylthiourea and

### EXAMPLE 11

### N-[(3aS*,6R*,7aS*)-1-benzyl-3a-(3,4-dimethoxyphenyl)octahydro-1H-indol-6-yl]-N′-ethylthiourea

The compounds were synthesised (starting with 705 mg of the mixture of amines) and purified in an analogous method to that described in Example 14 and 15 to give:
N-[(3aS*,6R*,7aS*)-1-benzyl-3a-(3,4-dimethoxyphenyl)octahydro-1H-indol-6-yl]-N′-ethylthiourea (80 mg, 9%)

MS (ESI+) for C₂₆H₃₅N₃O₂S: m/z 454.0 (M+1)

HRMS (EI) calcd C₂₆H₃₅N₃O₂S: 453.2450, found 453.2472
N-[(3aS*,6S*,7aS*)-1-benzyl-3a-(3,4-dimethoxyphenyl)octahydro-1H-indol-6-yl]-N′-ethylthiourea (200 mg, 23%)

MS (ESI+) for C₂₆H₃₅N₃O₂S: m/z 454.0 (M+1)

HRMS (EI) calcd C₂₆H₃₅N₃O₂S: 453.2435, found 453.2450

### EXAMPLE 12

### N-[(3 aS*,6R*,7aS*)-1-benzyl-3a-(3,4-dimethoxyphenyl)octahydro-1H-indol-6-yl]-N′-butylthiourea and

### EXAMPLE 13

### N-[(3aS*,6S*,7aS*)-1-benzyl-3a-(3,4-dimethoxyphenyl)octahydro-1H-indol-6-yl]-N′-butylthiourea

Compounds were prepared and purified in an analougous method to that described in Example 14 and 15 to give:
N-[(3aS*,6R*,7aS*)-1-benzyl-3a-(3,4-dimethoxyphenyl)octahydro-1H-indol-6-yl]-N′-butylthiourea (14 mg, 13%)

MS (ESI+) for C₂₈H₃₉N₃O₂S: m/z 482.1 (M+1)

HRMS (EI) calcd C₂₈H₃₉N₃O₂S: 481.2763, found 481.2765
N-[(3aS*,6S*,7aS*)-1-benzyl-3a-(3,4-dimethoxyphenyl)octahydro-1H-indol-6-yl]-N′-butylthiourea (30 mg, 28%)

MS (ESI+) for C₂₈H₃₉N₃O₂S: m/z 482.1 (M+1)

HRMS (EI) calcd C₂₈H₃₉N₃O₂S: 481.2763, found 481.2742

### EXAMPLE 14

### N-benzyl-N′-[(3aS*,6R*,7aS*)-1-benzyl-3a-(3,4-dimethoxyphenyl)octahydro-1H-indol-6-yl]thiourea and

### EXAMPLE 15

### N-benzyl-N′-[(3aS*,6S*,7aS*)-1-benzyl-3a-(3,4-dimethoxyphenyl)octahydro-1H-indol-6-yl]thiourea

The mixture of cis and trans amines from Comparative Example 5 and Comparative Example 6 (82.5 mg, 225 μmol) was dissolved in dichloromethane (5 mL). Benzyl isothiocyanate (39 μL, 43.9 mg, 294 μmol) was added and the mixture stirred at room temperature for 16 hours. The solvent was then removed, and the crude reaction mixture chromatographed (SiO₂, petroleum ether/ethyl acetate 5:2 as eluent) to give:
N-benzyl-N′-[(3aS*,6R*,7aS*)-1-benzyl-3a-(3,4-dimethoxyphenyl)octahydro-1H-indol-6-yl]thiourea (10 mg, 9%):

MS (ESI+) for C₃₁H₃₇N₃O₂S: m/z 516.2 (M+1)

HRMS (EI) calcd C₃₁H₃₇N₃O₂S: 515.2606, found 516.2602
N-benzyl-N′-[(3aS*,6S*,7aS*)-1-benzyl-3a-(3,4-dimethoxyphenyl)octahydro-1H-indol-6-yl]thiourea (25 mg, 22%):

MS (ESI+) for C₃₁H₃₇N₃O₂S: m/z 516.2 (M+1)

HRMS (EI) calcd C₃₁H₃₇N₃O₂S: 515.2606, found 516.2623

### EXAMPLE 16

### N-[(3aS*,6S*,7aS*)-1-benzyl-3a-(3,4-dimethoxyphenyl)octahydro-1H-indol-6-yl]-N′-ethylurea

The compound was synthesised and purified an analougous method to that described in Example 14 and 15 to give:
N-[(3aS*,6S*,7aS*)-1-benzyl-3a-(3,4-dimethoxyphenyl)octahydro-1H-indol-6-yl]-N′-ethylurea (20 mg, 20%)

MS (ESI+) for C₂₆H₃₅N₃O₃: m/z 438.5 (M+1)

HRMS (EI) calcd C₂₆H₃₅N₃O₃: 437.2678, found 437.2670

### EXAMPLE 17

### tert-butyl (3aS*,6R*,7aS*)-6-{[(benzylamino)carbonothioyl]amino}-3a-(3,4-dimethoxyphenyl)octahydro-1H-indole-1-carboxylate and

### EXAMPLE 18

### tert-butyl (3aS*,6S*,7aS*)-6-{[(benzylamino)carbonothioyl]amino}-3a-(3,4-dimethoxyphenyl)octahydro-1H-indole-1-carboxylate

NH₄OAc (2 g) was added to a solution of tert-butyl (3aS*,7aS*)-3a-(3,4-dimethoxyphenyl)-6-oxooctahydro-1H-indole-1-carboxylate (Comparative Example 9, 2.4 g, 6.4 mmol) in MeOH (50 ml), and the solution was stirred at ambient temperature for 2 h before NaBH₃CN (400 mg) was added and the mixture stirred overnight. The mixture was diluted with 3 M NaOH (50 ml) and extracted with dichloromethane. The crude mixture of amines was used without further purifications.

Benzyl thioisocyanate (1.5 eqv.) was added to a solution of the amine mixture from above (0.1 g, 0.26 mmol) in CHCl₃ (50 ml) and the mixture was stirred at ambient temperature overnight. Trisamine-PS (1 g) was added and the mixture stirred for 4 h before filtration and separation of products by flash chromatography (silica, CHCl₃/MeOH/NH₃). First eluted. tert-butyl (3aS*,6R*,7aS*)-6-{[(benzylamino)carbonothioyl]amino}-3a-(3, 4-dimethoxyphenyl)octahydro-1H-indole-1-carboxylate: 215 mg.

¹H NMR (270 MHz, CDCl₃): δ ppm 1.13-1.53 (m, 10H), 1.65-1.99 (m, 4H), 1.99-2.23 (m, 1H), 2.92-3.31 (m, 1H), 3.31-3.66 (m, 3H), 3.77-3.94 (m, 6H), 3.96-4.15 (m, 1H), 4.74-5.01 (m, 1H), 5.79-5.96 (m, 1H), 6.74-6.91 (m, 3H), 7.18-7.34 (m, 3H), 7.34-7.44 (m, 1H), 7.44-7.70 (m, 1H). (ESI+) m/z 526 (M+1).

HRMS (EI) Calc for C₃₂H₃₆F₃N₃O₃S: 525.2661; found 525.2662.

Relative configuration was determend by NMR.
Second eluted: tert-butyl (3aS*,6S*,7aS*)-6-([(benzylamino)carbonothioyl]amino)-3a-(3,4-dimethoxyphenyl)octahydro-1H-indole-1-carboxylate: 300 mg

### EXAMPLE 19

### N-benzyl-N′-[(3aS*,6R*,7aS*)-3a-(3,4-dimethoxyphenyl)octahydro-1H-indol-6-yl]thiourea

To a solution of Example 17 (600 mg, 1.14 mmol) in DCM (20 mL) was added trifluoroacetic acid (20 mL) and the resulting solution was stirred at rt during 5 min. The volatiles were evaporated under reduced pressure and the residue was partitioned between EtOAc (5 mL) and NaOH (aq., 1M, 2 mL). The water phase was extracted with EtOAc and the organic phases were washed with saturated aqueous NaCl (2 mL), dried (MgSO₄) and evaporated to give the title compound (0.44 g, 91%) as a colorless oil.

¹H NMR (270 MHz, CDCl₃): δ ppm 1.01-1.15 (m, 1H), 1.15-1.42 (m, 1H), 1.56-1.68 (m, 1H), 1.72-1.89 (m, 2H), 1.90-2.15 (m, 4H), 2.20-2.33 (m, 1H), 2.34-2.45 (m, 1H), 3.09-3.22 (m, 1H), 3.22-3.40 (m, 1H), 3.77-3.97 (m, 6H), 4.17-4.39 (m, 1H), 4.59-4.94 (m, 2H), 5.67-6.16 (m, 1H), 6.65-6.94 (m, 3H), 7.18-7.40 (m, 5H). MS (ESI+) m/z 426 (M+1).

HRMS (EI) Calc for C₂₄H₃₁N₃O₂S: 425.2137; found 425.2157.

### EXAMPLE 20

### N-benzyl-N′-[(3aS*,6R*,7aS*)-3a-(3,4-dimethoxyphenyl)-1-(quinolin-3-ylmethyl)octahydro-1H-indol-6-yl]thiourea

Following the general procedure for reductive alkylation of the pyrrolidine moiety, Scheme E, using N-benzyl-N′-[(3 aS*,6R*,7aS*)-3a-(3,4-dimethoxyphenyl)octahydro-1H-indol-6-yl]thiourea (Example 19, 20 mg, 0.051 mmol) and quinoline-3-carbaldehyde (0.25 mmol), gave the title compound after preparative HPLC purification. ¹H NMR (270 MHz, CDCl₃): δ ppm 1.04-1.26 (m, 1H), 1.26-2.61 (m, 8H), 2.83-3.20 (m, 3H), 3.20-3.38 (m, 1H), 3.69-3.91 (m, 6H), 4.27-4.67 (m, 3H), 4.77-4.99 (m, 2H), 5.21-5.46 (m, 1H), 5.75-6.07 (m, 1H), 6.68-6.90 (m, 3H), 7.40-7.58 (m, 2H), 7.58-7.69 (m, 1H), 7.72-7.87 (m, 1H), 7.99-8.21 (m, 3H), 8.81-8.81-8.92 (m, 1H). MS (ESI+) m/z 567 (M+1). HRMS (EI) Calc for C₃₄H₃₈N₄O₂S: 566.2715; found 566.2695.

### EXAMPLE 21

### N-benzyl-N′-[(3aS*,6R*,7aS*)-3a-(3,4-dimethoxyphenyl)-1-[3-(trifluoromethyl)benzyl]octahydro-1H-indol-6-yl]thiourea

Following the general procedure for reductive alkylation of the pyrrolidine moiety, Scheme E, using N-benzyl-N′-[(3aS*,6R*,7aS*)-3a-(3,4-dimethoxyphenyl)octahydro-1H-indol-6-yl]thiourea (Example 19, 20 mg, 0.051 mmol) and 3-(trifluoromethyl)benzaldehyde (0.25 mmol), gave the title compound after preparative HPLC purification. 11H NMR (270 MHz, CDCl₃): δ ppm 1.01-1.17 (m, 1H), 1.24-1.59 (m, 3H), 1.59-1.85 (m, 3H), 1.85-2.51 (m, 4H), 2.81-3.24 (m, 3H), 3.67-3.92 (m, 6H), 4.10-4.33 (m, 1H), 4.33-4.70 (m, 3H), 5.20-5.57 (m, 1H), 5.72-6.05 (m, 2H), 6.66-6.91 (m, 3H), 7.19-7.85 (m, 7H). (ESI+) m/z 584 (M+1). HRMS (EI) Calc for C₃₂H₃₆F₃N₃O₂S: 583.2480; found 583.2487

### EXAMPLE 22

### N-benzyl-N′-{(3aS*,6R*,7aS*)-3a-(3,4-dimethoxyphenyl)-1-[4-(trifluoromethoxy)benzyl]octahydro-1H-indol-6-yl}thiourea

Following the general procedure for reductive alkylation of the pyrrolidine moiety, Scheme E, using N-benzyl-N′-[(3 aS*,6R*,7aS*)-3a-(3,4-dimethoxyphenyl)octahydro-1H-indol-6-yl]thiourea (Example 19, 20 mg, 0.051 mmol) and 4-(trifluoromethoxy)benzaldehyde (0.25 mmol), gave the title compound after preparative HPLC purification. ¹H NMR (270 MHz, CDCl₃): δ ppm 1.03-1.29 (m, 1H), 1.30-1.49 (m, 1H), 1.49-1.66 (m, 2H), 1.66-1.91 (m, 3H), 1.91-2.35 (m, 3H), 2.35-2.60 (m, 1H), 2.83-3.22 (m; 3H), 3.72-3.97 (m, 6H), 4.09-4.34 (m, 1H), 4.34-4.76 (m, 2H), 5.75-6.02 (m, 1H), 6.73-6.95 (m, 3H), 7.05-7.19 (m, 2H), 7.20-7.34 (m, 5H), 7.34-7.49 (m, 2H).

MS (ESI+) m/z 600 (M+1). HRMS (EI) Calc for C₃₂H₃₆F₃N₃O₃S: 599.2429; found 599.2443

### EXAMPLE 23

### N-benzyl-N′-[(3aS*,6R*,7aS*)-3a-(3,4-dimethoxyphenyl)-1-(3-phenylpropyl)octahydro-1H-indol-6-yl]thiourea

Following the general procedure for reductive alkylation of the pyrrolidine moiety, Scheme E, using N-benzyl-N′-[(3aS*,6R*,7aS*)-3a-(3,4-dimethoxyphenyl)octahydro-1H-indol-6-yl]thiourea (Example 19, 20 mg, 0.051 mmol) and 3-phenylpropanal (0.25 mmol), gave the title compound as a colorless after preparative HPLC purification. ¹H NMR (270 MHz, CDCl₃): δ ppm 0.95-1.42 (m, 3H), 1.42-1.87 (m, 6H), 1.87-2.35 (m, 5H), 2.35-3.04 (m, 4H), 3.07-3.31 (m, 1H), 3.65-3.97 (m, 6H), 4.35-4.80 (m, 2H), 5.28-6.01 (m, 1H), 6.60-6.91 (m, 3H), 6.91-7.44 (m, 10H). MS (ESI+) m/z 544 (M+1). HRMS (EI) Calc for C₃₃H₄₁N₃O₂S: 543.2919; found 543.2902.

### EXAMPLE 24

### N-benzyl-N′-[(3aS*,6R*,7aS*)-3a-(3,4-dimethoxyphenyl)-1-(pyridin-3-ylmethyl)octahydro-1H-indol-6-yl]thiourea

Following the general procedure for reductive alkylation of the pyrrolidine moiety, Scheme E, using N-benzyl-N′-[(3aS*,6R*,7aS*)-3a-(3,4-dimethoxyphenyl)octahydro-1H-indol-6-yl]thiourea (Example 19, 20 mg, 0.051 mmol) and pyridine-3-carbaldehyde (0.25 mmol), gave the title compound as a colorless oil after preparative HPLC purification. ¹H NMR (270 MHz, CDCl₃): δ ppm 1.01-1.40 (m, 3H), 1.43-1.60 (m, 2H), 1.67-1.78 (m, 3H), 1.91-2.04 (m, 1H), 2.04-2.26 (m, 2H), 2.37-2.50 (m, 1H), 2.83-2.99 (m, 1H), 2.99-3.14 (m, 2H), 3.62-3.69 (m, 1H), 3.76-3.87 (m, 7H), 4.12-4.27 (m, 1H), 4.36-4.62 (m, 2H), 5.77-5.97 (m, 1H), 6.71-6.71-6.84 (m, 3H), 6.96-7.00 (m, 1H), 7.38-7.42 (m, 1H), 7.71-7.85 (m, 5H), 8.40-8.45 (m, 1H), 8.47-8.53 (m, 1H). MS (ESI+) m/z 517 (M+1). HRMS (EI) Calc for C₃₀H₃₆N₄O₂S: 516.2559; found 516.2557.

### EXAMPLE 25

### N-benzyl-N′-[(3aS*,6R*,7aS*)-3a-(3,4-dimethoxyphenyl)-1-(4-methoxybenzyl)octahydro-1H-indol-6-yl]thiourea

Following the general procedure for reductive alkylation of the pyrrolidine moiety, Scheme E, using N-benzyl-N′-[(3aS*,6R*,7aS*)-3a-(3,4-dimethoxyphenyl)octahydro-1H-indol-6-yl]thiourea (Example 19, 20 mg, 0.051 mmol) and 4-methoxybenzaldehyde (0.25 mmol), gave the title compound as a colorless oil after preparative HPLC purification. ¹H NMR (270 MHz, CDCl₃): δ ppm 1.01-1.15 (m, 1H), 1.26-1.43 (m, 1H), 1.43-1.63 (m, 2H), 1.63-1.85 (m, 3H), 1.85-2.16 (m, 2H), 2.16-2.43 (m, 1H), 2.84-3.20 (m, 3H), 3.66-3.77 (m, 3H), 3.77-3.92 (m, 6H), 3.95-4.18 (m, 1H), 4.37-4.73 (m, 2H), 5.28-5.62 (m, 1H), 5.62-5.99 (m, 1H), 6.62-6.62-6.95 (m, 5H), 7.03-7.44 (m, 7H).

MS (ESI+) m/z 546 (M+1).

HRMS (EI) Calc for C₃₂H₃₉N₃O₃S: 545.2712; found 545.2712.

### EXAMPLE 26

### ethyl[(3aS*,6R*,7aS*)-6-{[(benzylamino)carbonothioyl]amino}-3a-(3,4-dimethoxyphenyl)octahydro-1H-indol-1-yl]acetate

Following the general procedure for reductive alkylation of the pyrrolidine moiety, Scheme E, using N-benzyl-N′-[(3aS*,6R*,7aS*)-3a-(3,4-dimethoxyphenyl)octahydro-1H-indol-6-yl]thiourea (Example 19, 20 mg, 0.051 mmol) and ethyl glyoxalate (0.25 mmol), gave the title compound as a colorless oil after preparative HPLC purification. ¹H NMR (270 MHz, CDCl₃): δ ppm 1.04-1.14 (m, 3H), 1.18-1.26 (m, 11H), 1.34-1.43 (m, 11H), 1.43-1.54 (m, 2H), 1.68-1.76 (m, 1H), 1.76-1.85 (m, 2H), 1.89-2.13 (m, 3H), 2.45-2.69 (m, 1H), 2.98-3.50 (m, 4H), 3.64-3.78 (m, 1H), 3.78-3.83 (m, 6H), 3.86-3.97 (m, 1H), 3.97-4.20 (m, 1H), 4.57-4.94 (m, 2H), 5.44-4.94-5.77 (m, 1H), 6.66-6.85 (m, 3H), 7.22-7.35 (m, 5H). MS (ESI+) m/z 512 (M+1).

HRMS (EI) Calc for C₂₈H₃₇N₃O₄S: 511.2505; found 511.2518.

### EXAMPLE 27

### N-benzyl-N′-[(3aS*,6R*,7aS*)-3a-(3,4-dimethoxyphenyl)-1-(4-nitrobenzyl)octahydro-1H-indol-6-yl]thiourea

Following the general procedure for reductive alkylation of the pyrrolidine moiety, Scheme E, using N-benzyl-N′-[(3aS*,6R*,7aS*)-3a-(3,4-dimethoxyphenyl)octahydro-1H-indol-6-yl]thiourea (Example 19, 20 mg, 0.051 mmol) and 4-nitrobenzaldehyde (0.25 mmol), gave the title compound as a colorless oil after preparative HPLC purification. ¹H NMR (270 MHz, CDCl₃): δ ppm 1.04-1.84 (m, 9H), 1.89-2.28 (m, 3H), 2.33-2.57 (m, 1H), 2.72-3.00 (m, 1H), 3.00-3.23 (m, 2H), 3.70-3.89 (m, 6H), 4.14-4.67 (m, 4H), 5.19-5.37 (m, 1H), 5.67-6.02 (m, 1H), 6.51-6.62 (m, 1H), 6.69-6.87 (m, 3H), 7.20-7.36 (m, 3H), 7.48-7.64 (m, 2H), 7.70-7.85 (m, 1H), 8.04-8.14 (m, 3H), 8.15-8.32 (m, 1H). MS (ESI+) m/z 544 (M+1).

### EXAMPLE 28

### N-benzyl-N′-[(3aS*,6R*,7aS*)-3a-(3,4-dimethoxyphenyl)-1-pentyloctahydro-1H-indol-6-yl]thiourea

Following the general procedure for reductive alkylation of the pyrrolidine moiety, Scheme E, using N-benzyl-N′-[(3aS*,6R*,7aS*)-3a-(3,4-dimethoxyphenyl)octahydro-1H-indol-6-yl]thiourea (Example 19, 20 mg, 0.051 mmol) and pentanal (0.25 mmol), gave the title compound as a colorless oil after preparative HPLC purification.

¹H NMR (270 MHz, CDCl₃): δ ppm 0.70-0.91 (m, 3H), 0.91-1.85 (m, 9H), 1.85-2.09 (m, 3H), 2.09-2.30 (m, 2H), 2.59-2.78 (m, 1H), 2.78-2.94 (m, 1H), 3.04-3.24 (m, 1H), 3.70-3.88 (m, 6H), 4.48-4.81 (m, 1H), 5.64-5.88 (m, 1H), 6.64-6.87 (m, 3H), 7.20-7.35 (m, 5H). MS (ESI+) m/z 496 (M+1). HRMS (EI) Calc for C₂₉H₄₁N₃O₂S: 495.2919; found 495.2929.

### EXAMPLE 29

### N-benzyl-N′-[(3aS*,6S*,7aS*)-3a-(3,4-dimethoxyphenyl)octahydro-1H-indol-6-yl]thiourea

To a solution of tert-butyl (3aS*,6S*,7aS*)-6-{[(benzylamino)carbonothioyl]amino}-3a-(3,4-dimethoxyphenyl)octahydro-1H-indole-1-carboxylate (Example 18, 10 mg) in DCM (2 mL) was added trifluoroacetic acid (2 mL) and the resulting solution was stirred at rt during 5 min. The volatiles were evaporated under reduced pressure and the residue was partitioned between EtOAc (5 mL) and NaOH (aq., 1M, 2 mL). The water phase was re-extracted with EtOAc and the organic phases were washed with saturated aqueous NaCl (2 mL), dried (MgSO₄) and evaporated to give the title compound as a colourless oil. ¹H NMR (500 MHz, CDCl₃): δ ppm 1.34-1.46 (m, 1H), 1.61-2.08 (m, 9H), 2.75-3.04 (m, 2H), 3.69-3.79 (m, 1H), 3.79-3.95 (m, 6H), 4.25-4.79 (m, 3H), 6.09-6.61 (m, 1H), 6.78-6.85 (m, 3H), 7.27-7.39 (m, 4H), 8.78-9.64 (m, 1H). MS (ESI+): m/z 426 (M+1).

### EXAMPLE 30

### N-allyl-N′-[(3aS*,6R*,7aS*)-1-benzyl-3a-(3,4-dimethoxyphenyl)octahydro-1H-indol-6-yl]urea trifluoroacetate

Reagent: allyl isocyanate

Synthetic procedure: Scheme A

Yield: 12.7 mg

Measured mass: 449.2669

Calc. mass: 449.2678

### EXAMPLE 31

### N-[(3aS*,6R*,7aS*)-1-benzyl-3a-(3,4-dimethoxyphenyl)octahydro-1H-indol-6-yl]-N′-(2-furylmethyl)thiourea trifluoroacetate

Reagent: 2-(isothiocyanatomethyl)furan

Synthetic procedure: Scheme A

Yield: 0.9 mg

Measured mass: 502.2395

Calc. mass: 505.2399

### EXAMPLE 32

### N-[(3aS*,6R*,7aS*)-1-benzyl-3a-(3,4-dimethoxyphenyl)octahydro-1H-indol-6-yl]-N′-(2-morpholin-4-ylethyl)thiourea trifluoroacetate

Reagent: 4-(2-isothiocyanatoethyl)morpholine

Synthetic procedure: Scheme A

Yield: 16.9 mg

Measured mass: 538.3000

Calc. mass: 538.2978

### EXAMPLE 33

### N-benzyl-N′-[(3aS*,6R*,7aS*)-1-benzyl-3a-(3,4-dimethoxyphenyl)octahydro-1H-indol-6-yl]urea trifluoroacetate

Reagent: (isocyanatomethyl)benzene

Synthetic procedure: Scheme A

Yield: 17.0 mg

Measured mass: 499.2851

Calc. mass: 499.2836

### EXAMPLE 34

### N-[(3aS*,6R*,7aS*)-1-benzyl-3a-(3,4-dimethoxyphenyl)octahydro-1H-indol-6-yl]-N′-(3-methoxypropyl)thiourea trifluoroacetate

Reagent: 1-isothiocyanato-3-methoxypropane

Synthetic procedure: Scheme A

Yield: 12.8 mg

Measured mass: 497.2706

Calc. mass: 497.2712

### EXAMPLE 35

### N-[(3aS*,6R*,7aS*)-1-benzyl-3a-(3,4-dimethoxyphenyl)octahydro-1H-indol-6-yl]-N′-(cyclopropylmethyl)thiourea trifluoroacetate

Reagent: cyclopropylmethyl isothiocyanate

Synthetic procedure: Scheme A

Yield: 14.0 mg

Measured mass: 479.2615

Calc. mass: 479.2606

### EXAMPLE 36

### N-allyl-N′-[(3aS*,6R*,7aS*)-1-benzyl-3a-(3,4-dimethoxyphenyl)octahydro-1H-indol-6-yl]thiourea

Reagent: allyl isothiocyanate

Synthetic procedure: Scheme A

Yield: 1.1 mg

Measured mass: 465.2444

Calc. mass: 465.2450

### EXAMPLE 37

### N-[(3aS*,6R*,7aS*)-1-benzyl-3a-(3,4-dimethoxymhenyl)octahydro-1H-indol-6-yl]-N′-isobutylthiourea trifluoroacetate

Reagent: isobutyl isothiocyanate

Synthetic procedure: Scheme A

Yield: 14.1 mg

Measured mass: 481.2753

Calc. mass: 481.2763

### EXAMPLE 38

### N-[(3 aS*,6R*,7aS*)-1-benzyl-3a-(3,4-dimethoxyphenyl)octahydro-1H-indol-6-yl]-N′-(2-phenylethyl)thiourea trifluoroacetate

Reagent: 2-phenylethyl isothiocyanate

Synthetic procedure: Scheme A

Yield: 12 mg

Measured mass: 529.2755

Calc. mass: 529.2763

### EXAMPLE 39

### ethyl N-({[(3aS*,6R*,7aS*)-1-benzyl-3a-(3,4-dimethoxyphenyl)octahydro-1H-indol-6-yl]amino}carbonyl)-beta-alaninate trifluoroacetate

Reagent: ethyl 3-isocyanatopropionate

Synthetic procedure: Scheme A

Yield: 9.0 mg

Measured mass: 509.2882

Calc. mass: 509.2890

### EXAMPLE 40

### N-butyl-N′-[(3aS*,6R*,7aS*)-3a-(3,4-dimethoxyphenyl)-1-ethyloctahydro-1H-indol-6-yl]thiourea trifluoroacetate

To a solution of mixture of amines, intermediate from Example 17 and 18 (2.2 g, 5.84 mmol) in dichloromethane (50 ml) was added n-butyl thioisocyanate (1.02 g, 8.8 mmol) and the mixture was stirred orvernight. PS-trisamine (2 g) was added and mixture was filtered and purified by flash chromatography using 20-70% EtOAc in hexanes. Yield: 800 mg of cis-compound.

The BOC-protected compound from above was deproteced by stirring in dichloromethane (10 ml) and trifluoroacetic acid (10 ml) for 30 min. The mixture was concentrated and redissolved in chloroform. PS-trisamine was added and the mixture filtered. The crude material was used without further purifications.

Following the general procedure for reductive alkylation of the pyrrolidine moiety, Scheme E, using the deprotected amine from above (20 mg, 0.051 mmol) and acetaldehyde (0.25 mmol), gave the title compound as a colorless oil after preparative HPLC purification. ¹H NMR (270 MHz, CDCl₃): δ ppm 0.86-0.98 (m, 3H), 1.25-0.98 1.40 (m, 4H), 1.40-1.57 (m, 5H), 1.67-1.95 (m, 4H), 2.08-2.35 (m, 3H), 2.35-2.49 (m, 1H), 2.61-2.78 (m, 1H), 3.34-3.50 (m, 2H), 3.63-3.78 (m, 1H), 3.80-3.92 (m, 7H), 4.05-4.16 (m, 1H), 4.41-4.60 (m, 2H), 6.93-7.04 (m, 3H). MS (ESI+) m/z 420 (M+1). HRMS (EI) Calc for C₂₃H₃₇N₃O₂S: 419.2606; found 419.2604.

### EXAMPLE 41

### N-butyl-N′-[(3aS*,6R*,7aS*)-3a-(3,4-dimethoxyphenyl)-1-propyloctahydro-1H-indol-6-yl]thiourea trifluoroacetate

Following the general procedure for reductive alkylation of the pyrrolidine moiety, Scheme E, using deprotected amine, intermediate from Example 40 (20 mg, 0.051 mmol) and propanal (0.25 mmol), gave the title compound as a colorless oil after preparative HPLC purification. ¹H NMR (270 MHz, CDCl₃): δ ppm 0.86-1.01 (m, 3H), 1.24-1.61 (m, 5H), 1.61-2.04 (m, 4H), 2.16-2.39 (m, 2H), 2.39-2.59 (m, 1H), 2.67-2.88 (m, 1H), 3.05-3.26 (m, 3H), 3.37-3.66 (m, 2H), 3.74-4.00 (m, 7H), 4.11-4.24 (m, 1H), 4.44-4.63 (m, 2H), 6.91-7.06 (m, 3H), 7.20-7.49 (m, 5H). MS (ESI+) m/z 434 (M+1). HRMS (EI) Calc for C₂₄H₃₉N₃O₂S: 433.2763; found 433.2775.

### EXAMPLE 42

### N-butyl-N′-[(3aS*,6R*,7aS*)-3a-(3,4-dimethoxyphenyl)-1-isobutyloctahydro-1H-indol-6-yl]thiourea trifluoroacetate

Following the general procedure for reductive alkylation of the pyrrolidine moiety, Scheme E, using deprotected amine, intermediate from Example 40 (20 mg, 0.051 mmol) and isobutyraldehyde (0.25 mmol), gave the title compound as a colorless after preparative HPLC purification. ¹H NMR (270 MHz, CDCl₃): δ ppm 0.85-0.85-1.01 (m, 3H), 1.09-1.24 (m, 5H), 1.24-1.62 (m, 6H), 1.62-2.09 (m, 4H), 2.09-2.47 (m, 5H), 2.65-2.83 (m, 1H), 3.07-3.20 (m, 1H), 3.34-3.53 (m, 3H), 3.78-3.90 (m, 6H), 3.90-4.08 (m, 1H), 4.08-4.21 (m, 1H), 4.40-4.61 (m, 2H), 6.88-7.10 (m, 3H). MS (ESI+) m/z 448 (M+1). HRMS (EI) Calc for C₂₅H₄₁N₃O₂S: 447.2919; found 447.2808.

### EXAMPLE 43

### N-butyl-N′-[(3aS*,6R*,7aS*)-3a-(3,4-dimethoxyphenyl)-1-pentyloctahydro-1H-indol-6-yl]thiourea trifluoroacetate

Following the general procedure for reductive alkylation of the pyrrolidine moiety, Scheme E, using deprotected amine, intermediate from Example 40 (20 mg, 0.051 mmol) and pentanal (0.25 mmol), gave the title compound as a colorless oil after preparative HPLC purification. ¹H NMR (270 MHz, CDCl₃): δ ppm 0.85-1.03 (m, 6H), 1.22-1.59 (m, 10H), 1.61-2.00 (m, 7H), 2.10-2.33 (m, 2H), 2.33-2.49 (m, 1H), 2.64-2.81 (m, 1H), 3.08-3.25 (m, 1H), 3.35-3.50 (m, 2H), 3.50-3.70 (m, 1H), 3.74-3.89 (m, 7H), 4.06-4.16 (m, 1H), 4.38-4.60 (m, 1H), 6.92-7.02 (m, 3H). MS (ESI+) m/z 462 (M+1). HRMS (EI) Calc for C₂₆H₄₃N₃O₂S: 461.3076; found 461.3059.

### EXAMPLE 44

### N-butyl-N′-[(3aS*,6R*,7aS*)-3a-(3,4-dimethoxyphenyl)-1-(2-phenylethyl)octahydro-1H-indol-6-yl]thiourea trifluoroacetate

Following the general procedure for reductive alkylation of the pyrrolidine moiety, Scheme E, using deprotected amine, intermediate from Example 40 (20 mg, 0.051 mmol) and phenylacetaldehyde (0.25 mmol), gave the title compound as a colorless oil after preparative HPLC purification. ¹H NMR (270 MHz, CDCl₃): δ ppm 0.84-1.00 (m, 3H), 1.26-1.58 (m, 6H), 1.61-1.82 (m, 1H), 1.82-2.03 (m, 2H), 2.15-2.37 (m, 3H), 2.38-2.54 (m, 1H), 2.68-2.87 (m, 1H), 3.39-3.65 (m, 3H), 3.73-4.00 (m, 11H), 4.13-4.22 (m, 2H), 4.46-4.61 (m, 2H), 6.92-7.04 (m, 3H), 7.21-7.48 (m, 5H). MS (ESI+) m/z 496 (M+1). HRMS (EI) Calc for C₂₉H₄₁N₃O₂S: 495.2919; found 495.2914.

### EXAMPLE 45

### Ethyl 2-{[(3aS*,6R*,7aS*)-6-{[(butylamino)carbonothioyl]amino}-3a-(3,4-dimethoxyphenyl)octahydro-1H-indol-1-yl]methyl}cyclopronanecarboxylate trifluoroacetate

Following the general procedure for reductive alkylation of the pyrrolidine moiety, Scheme E, using deprotected amine, intermediate from Example 40 (20 mg, 0.051 mmol) and 2-formyl-cyclopropanecarboxylic acid ethyl ester (0.25 mmol), gave the title compound as a colorless oil in after preparative HPLC purification. ¹H NMR (270 MHz, CDCl₃): δ ppm 0.82-0.98 (m, 3H), 1.05-1.42 (m, 9H), 1.42-1.60 (m, 2H), 1.66-2.02 (m, 5H), 2.12-2.36 (m, 2H), 2.36-2.51 (m, 1H), 2.59-2.80 (m, 1H), 3.14-3.27 (m, 2H), 3.47-3.74 (m, 3H), 3.74-4.02 (m, 7H), 4.05-4.23 (m, 3H), 4.36-4.57 (m, 2H), 6.89-7.05 (m, 3H).

MS (ESI+) m/z 518 (M+1). HRMS (EI) Calc for C₂₈H₄₃N₃O₄S: 517.2974; found 517.2973.

### EXAMPLE 46

### N-butyl-N′-[(3aS*,6R*,7aS*)-3a-(3,4-dimethoxyphenyl)-1-(3-furylmethyl)octahydro-1H-indol-6-yl]thiourea trifluoroacetate

Following the general procedure for reductive alkylation of the pyrrolidine moiety, Scheme E, using deprotected amine, intermediate from Example 40 (20 mg, 0.051 mmol) and furan-3-carbaldehyde (0.25 mmol), gave the title compound as a colorless oil after preparative HPLC purification. ¹H NMR (270 MHz, CDCl₃): δ ppm 0.84-1.01 (m, 3H), 1.24-1.61 (m, 5H), 1.61-2.00 (m, 4H), 2.03-2.34 (m, 3H), 2.34-2.48 (m, 1H), 2.68-2.82 (m, 1H), 3.25-3.47 (m, 1H), 3.47-3.64 (m, 2H), 3.64-3.81 (m, 2H), 3.81-3.89 (m, 6H), 4.09-4.18 (m, 1H), 4.29-4.44 (m, 1H), 4.60-4.71 (m, 1H), 6.70-6.80 (m, 1H), 6.89-7.03 (m, 3H), 7.63-7.71 (m, 11H), 7.85-7.93 (m, 11H). MS (ESI+) m/z 472 (M+1). HRMS (EI) Calc for C₂₆H₃₇N₃O₃S: 471.2556; found 471.2569.

### EXAMPLE 47

### N-butyl-N′-{(3aS*,6R*,7aS*)-3a-(3,4-dimethoxyphenyl)-1-[(1-methyl-1H-pyrrol-2-yl)methyl]octahydro-1H-indol-6-yl}thiourea trifluoroacetate

Following the general procedure for reductive alkylation of the pyrrolidine moiety, Scheme E, using deprotected amine, intermediate from Example 40 (20 mg, 0.051 mmol) and 1-methyl-1H-pyrrole-2-carbaldehyde (0.25 mmol), gave the title compound as a colorless oil after preparative HPLC purification. ¹H NMR (270 MHz, CDCl₃): δ 0.86-0.97 (m, 3H), 1.25-1.42 (m, 2H), 1.42-1.57 (m, 3H), 1.70-1.98 (m, 5H), 2.14-2.32 (m, 3H), 2.32-2.56 (m, 3H), 2.67-2.82 (m, 2H), 3.27-3.47 (m, 3H), 3.58-3.73 (m, 2H), 3.79-3.89 (m, 6H), 4.15-4.22 (m, 1H), 4.43-4.54 (m, 1H), 6.11-6.16 (m, 1H), 6.46-6.51 (m, 1H), 6.84-6.89 (m, 1H), 6.94-7.01 (m, 3H), 7.31-7.31-7.42 (m, 1H). MS(ESI+) m/z 485 (M+1). HRMS (EI) Calc for C₂₇H₄₀N₄O₂S: 484.2872; found 484.2880.

### EXAMPLE 48

### N-butyl-N′-{(3aS*,6R*,7aS*)-3a-(3,4-dimethoxyphenyl)-1-[(5-methyl-2-furyl)methyl]octahydro-1H-indol-6-yl}thiourea trifluoroacetate

Following the general procedure for reductive alkylation of the pyrrolidine moiety, Scheme E, using deprotected amine, intermediate from Example 40 (20 mg, 0.051 mmol) and 5-methyl-furan-2-carbaldehyde (0.25 mmol), gave the title compound as a colorless oil after preparative HPLC purification. ¹H NMR (270 MHz, CDCl₃): δ ppm 0.88-0.99 (m, 3H), 1.25-1.58 (m, 5H), 1.62-1.98 (m, 3H), 2.02-2.18 (m, 1H), 2.19-2.32 (m, 1H), 2.32-2.38 (m, 3H), 2.38-2.57 (m, 2H), 3.16-3.49 (m, 2H), 3.53-3.69 (m, 2H), 3.70-3.81 (m, 1H), 3.81-3.90 (m, 6H), 4.08-4.19 (m, 1H), 4.38-4.52 (m, 1H), 4.52-4.71 (m, 3H), 6.10-6.18 (m, 1H), 6.65-6.72 (m, 1H), 6.87-7.03 (m, 3H). MS (ESI+) m/z 486 (M+1). HRMS (EI) Calc for C₂₇H₃₉N₃O₃S: 485.2712; found 485.2721.

### EXAMPLE 49

### N-butyl-N′-[(3aS*,6R*,7aS*)-3a-(3,4-dimethoxyphenyl)-1-(3-thienylmethyl)octahydro-1H-indol-6-yl]thiourea trifluoroacetate

Following the general procedure for reductive alkylation of the pyrrolidine moiety, Scheme E, using deprotected amine, intermediate from Example 40 (20 mg, 0.051 mmol) and thiophene-3-carbaldehyde (0.25 mmol), gave the title compound as a colorless oil after preparative HPLC purification. ¹H NMR (270 MHz, CDCl₃): δ ppm 0.87-1.01 (m, 3H), 1.20-1.62 (m, 6H), 1.62-2.03 (m, 4H), 2.03-2.31 (m, 3H), 2.31-2.48 (m, 1H), 2.59-2.77 (m, 1H), 3.51-3.76 (m, 2H), 3.79-3.90 (m, 7H), 4.09-4.23 (m, 1H), 4.39-4.59 (m, 2H), 4.73-4.83 (m, 1H), 6.87-7.04 (m, 3H), 7.34-7.45 (m, 1H), 7.56-7.67 (m, 1H), 7.78-7.89 (m, 1H). MS (ESI+) m/z 488 (M+1). HRMS (EI) Calc for C₂₆H₃₇N₃O₂S₂: 487.2326; found 487.2327.

### EXAMPLE 50

### N-butyl-N′-{(3aS*,6R*,7aS*)-3a-(3,4-dimethoxyphenyl)-1-[(4-methyl-1H-imidazol-5-yl)methyl]octahydro-1H-indol-6-yl}thiourea trifluoroacetate

Following the general procedure for reductive alkylation of the pyrrolidine moiety, Scheme E, using deprotected amine, intermediate from Example 40 (20 mg, 0.051 mmol) and 5-Methyl-3H-imidazole-4-carbaldehyde (0.25 mmol), gave the title compound as a colorless oil after preparative HPLC purification. ¹H NMR (270 MHz, CDCl₃): δ ppm 0.86-1.01 (m, 3H), 1.23-1.60 (m, 6H), 1.66-2.08 (m, 3H), 2.12-2.34 (m, 2H), 2.34-2.48 (m, 1H), 2.48-2.59 (m, 4H), 2.60-2.79 (m, 1H), 3.16-3.64 (m, 3H), 3.68-3.95 (m, 8H), 4.19-4.35 (m, 1H), 4.41-4.67 (m, 2H), 6.90-7.08 (m, 3H), 8.81-8.86 (m, 1H).

MS (ESI+) m/z 486 (M+1). HRMS (EI) Calc for C₂₆H₃₉N₅O₂S: 485.2824; found 485.2839.

### EXAMPLE 51

### N-butyl-N′-[(3aS*,6R*,7aS*)-3a-(3,4-dimethoxyphenyl)-1-(3-phenylpropyl)octahydro-1H-indol-6-yl]thiourea trifluoroacetate

Following the general procedure for reductive alkylation of the pyrrolidine moiety, Scheme E, using deprotected amine, intermediate from Example 40 (20 mg, 0.051 mmol) and 3-phenylpropanal (0.25 mmol), gave the title compound as a colorless oil after preparative HPLC purification. ¹H NMR (270 MHz, CDCl₃): δ ppm 0.86-1.00 (m, 3H), 1.24-1.59 (m, 5H), 1.62-1.97 (m, 3H), 2.10-2.33 (m, 4H), 2.33-2.48 (m, 1H), 2.61-2.84 (m, 3H), 3.12-3.47 (m, 5H), 3.53-3.73 (m, 1H), 3.73-3.90 (m, 8H), 4.04-4.16 (m, 1H), 4.41-4.59 (m, 1H), 6.91-7.02 (m, 3H), 7.16-7.37 (m, 5H). MS (ESI+) m/z 510 (M+1). HRMS (EI) Calc for C₃₀H₄₃N₃O₂S: 509.3076; found 509.3068.

### EXAMPLE 52

### N-[(3aS*,6R*,7aS*)-1-benzyl-3a-(3,4-dimethoxyphenyl)octahydro-1H-indol-6-yl]-N′-(pyridin-4-ylmethyl)urea trifluoroacetate

Reagent: 4-(aminomethyl)pyridine

Synthetic procedure: Scheme B

Yield: 2.2 mg

Measured mass: 500.2781

Calc. mass: 500.2787

### EXAMPLE 53

### N′-[(3 aS*,6R*,7aS*)-1-benzyl-3a-(3,4-dimethoxyphenyl)octahydro-1H-indol-6-yl]-N-ethyl-N-methylurea trifluoroacetate

Reagent: n-ethylmethylamine

Synthetic procedure: Scheme B

Yield: 1.8 mg

Measured mass: 451.2837

Calc. mass: 451.2835

### EXAMPLE 54

### N-[(3aS*,6R*,7aS*)-3a-(3,4-dimethoxyphenyl)-1-methyloctahydro-1H-indol-6-yl]-N′-(4-fluorobenzyl)thiourea trifluoroacetate

Reagent: 4-fluorobenzyl isothiocyanate

Synthetic procedure: Scheme C

Yield: 3.9 mg

Measured mass: 457.2197

Calc. mass: 457.2199

### EXAMPLE 55

### N-[(3aS*,6R*,7aS*)-3a-(3,4-dimethoxyphenyl)-1-methyloctahydro-1H-indol-6-yl]-N′-[2-(2-thienyl)ethyl]urea trifluoroacetate

Reagent: 2-(thien-2-yl)ethyl isocyanate

Synthetic procedure: Scheme C

Yield: 2.8 mg

Measured mass: 443.2258

Calc. mass: 443.2243

### EXAMPLE 56

### N-[(3aS*,6R*,7aS*)-3a-(3,4-dimethoxyphenyl)-1-methyloctahydro-1H-indol-6-yl]-N′-(2-phenylethyl)urea trifluoroacetate

Reagent: phenethyl isocyanate

Synthetic procedure: Scheme C

Yield: 1.7 mg

Measured mass: 437.2671

Calc. mass: 437.2678

### EXAMPLE 57

### N-[(3aS*,6R*,7aS*)-3a-(3,4-dimethoxyphenyl)-1-methyloctahydro-1H-indol-6-yl]-N′-(4-methoxybenzyl)thiourea trifluoroacetate

Reagent: 4-methoxybenzyl isothiocyanate

Synthetic procedure: Scheme C

Yield: 2.8 mg

Measured mass: 469.2403

Calc. mass: 469.2399

### EXAMPLE 58

### N-allyl-N′-[(3aS*,6R*7aS*)-3a-(3,4-dimethoxyphenyl)-1-methyloctahydro-1H-indol-6-yl]urea trifluoroacetate

Reagent: allyl isocyanate

Synthetic procedure: Scheme C

Yield: 3.9 mg

Measured mass: 373.2357

Calc. mass: 373.2365

### EXAMPLE 59

### N-[(3aS*,6R*,7aS*)-3a-(3,4-dimethoxyphenyl)-1-methyloctahydro-1H-indol-6-yl-N′-hexylthiourea trifluoroacetate

Reagent: n-hexyl isothiocyanate

Synthetic procedure: Scheme C

Yield: 6.2 mg

Measured mass: 433.2746

Calc. mass: 433.2763

### EXAMPLE 60

### N-[(3aS*,6R*,7aS*)-3a-(3,4-dimethoxyphenyl)-1-methyloctahydro-1H-indol-6-yl]-N′-(3-methylbenzyl)urea trifluoroacetate

Reagent: 3-methylbenzyl isocyanate

Synthetic procedure: Scheme C

Yield: 7.3 mg

Measured mass: 437.2684

Calc. mass: 437.2678

### EXAMPLE 61

### N-[(3aS*,6R*,7aS*)-3a-(3,4-dimethoxyhenyl)-1-methyloctahydro-1H-indol-6-yl]-N′-(4-methoxybenzyl)urea trifluoroacetate

Reagent: 4-methoxybenzyl isocyanate

Synthetic procedure: Scheme C

Yield: 6.3 mg

Measured mass: 453.2633

Calc. mass: 453.2628

### EXAMPLE 62

### N-benzyl-N′-[(3aS*,6S*,7aS*)-3a-(3,4-dimethoxyphenyl)-1-methyloctahydro-1H-indol-6-yl]guanidine trifluoroacetate

N-benzyl-1H-pyrazole-1-carboximidamide hydrochloride (50 mg, 0.21 mmol), diispropylethylamine (0.03 ml, 0.21 mmol) and (3aS*,6S*,7aS*)-3a-(3,4-dimethoxyphenyl)-1-methyl-octahydro-1H-indol-6-amine, (Comparative Example 8, 60 mg, 0.21 mmol) were mixed in anhydrous DMF (1 ml) and heated at 100° C. for 2 hrs. The crude mixture was purified by preparative HPLC to give the title compound, 9 mg (8%)

HRMS (EI) calc: 422.2682 Found: 422.2676

### EXAMPLE 63

### N-butyl-N′-[(3aS*,6S*,7aS*)-3a-(3,4-dimethoxyphenyl)-1-methyloctahydro-1H-indol-6-yl]guanidine trifluoroacetate

N-butyl-1H-pyrazole-1-carboximidamide hydrochloride (43 mg, 0.21 mmol), diispropylethylamine (0.03 ml, 0.21 mmol) and (3aS*,6S*,7aS*)-3a-(3,4-dimethoxyphenyl)-1-methyl-octahydro-1H-indol-6-amine (Comparative Example 8, 60 mg, 0.21 mmol) were mixed in anhydrous DMF (1 ml) and heated at 100° C. for 2 hrs. The crude mixture was purified by preparative HPLC to give the title compound, 9 mg (9%) HRMS (EI) calc: 388.2838 Found: 388.2849.

### EXAMPLE 64

### N-[(3aS*,6S*,7aS*)-3a-(3,4-dimethoxyphenyl)-1-methyloctahydro-1H-indol-6-yl]-N′-pentylguanidine trifluoroacetate

N-pentyl-1H-pyrazole-1-carboximidamide hydrochloride (45 mg, 0.21 mmol), diispropylethylamine (0.03 ml, 0.21 mmol) and (3aS*,6S*,7aS*)-3a-(3,4-dimethoxyphenyl)-1-methyl-octahydro-1H-indol-6-amine (Comparative Example 8, 60 mg, 0.21 mmol) were mixed in anhydrous DMF (1 ml) and heated at 100° C. for 2 hrs. The crude mixture was purified by preparative HPLC to give the title compound, 9 mg (8%) HRMS (EI) calc: 402.2995 Found: 402.2991

### EXAMPLE 65

### N-butyl-N′-[(3aS*,6R*,7aS*)-3a-(3,4-dimethoxyphenyl)-1-methyloctahydro-1H-indol-6-yl]guanidine trifluoroacetate

N-butyl-1H-pyrazole-1-carboximidamide hydrochloride (6 mg, 0.03 mmol), diispropylethylamine (0.01 ml, 0.06 mmol) and (3aS*,6R*,7aS*)-3a-(3,4-dimethoxyphenyl)-1-methyloctahydro-1H-indol-6-amine (Comparative Example 7, 10 mg, 0.03 mmol) were mixed in anhydrous DMF (1 ml) and heated at 100° C. for 2 hrs. The crude mixture was purified by preparative HPLC to give the title compound, 1 mg (7%) HRMS (EI) calc: 388.2838 Found: 388.2856.

### EXAMPLE 66

### N-[(3aS*,6R*,7aS*)-3a-(3,4-dimethoxyphenyl)-1-methyloctahydro-1H-indol-6-yl]-N′-[4-(trifluoromethyl)benzyl]urea trifluoroacetate

Reagent: 1-(isocyanatomethyl)-4-(trifluoromethyl)benzene

Synthetic procedure: Scheme D

Measured mass: 491.2406

Calc. mass: 491.2396

### EXAMPLE 67

### N-(2,4-dichlorobenzyl)-N′-[(3aS*,6R*,7aS*)-3a-(3,4-dimethoxyphenyl)-1-methyloctahydro-1H-indol-6-yl]urea trifluoroacetate

Reagent: 2,4-dichloro-1-(isocyanatomethyl)benzene

Synthetic procedure: Scheme D

Measured mass: 491.1766

Calc. mass: 491.1746

### EXAMPLE 68

### N-[(3aS*,6R*,7aS*)-3a-(3,4-dimethoxyphenyl)-1-methyloctahydro-1H-indol-6-yl]-N′-(4-fluorobenzyl)urea trifluoroacetate

Reagent: 4-fluorobenzyl isocyanate

Synthetic procedure: Scheme D

Measured mass: 441.2438

Calc. mass: 441.2428

### EXAMPLE 69

### N-(4-bromobenzyl)-N′-[(3aS*,6R*,7aS*)-3a-(3,4-dimethoxyphenyl)-1-methyloctahydro-1H-indol-6-yl]urea trifluoroacetate

Reagent: 4-bromobenzyl isocyanate

Synthetic procedure: Scheme D

Measured mass: 501.1641

Calc. mass: 501.1627

### EXAMPLE 70

### N′-butyl-N-[(3aS*,6R*,7aS*)-3a-(3,4-dimethoxyphenyl)-1-methyloctahydro-1H-indol-6-yl]-N-methylthiourea trifluoroacetate and

### EXAMPLE 71

### N′-butyl-N-[(3aS*,6S*,7aS*)-3a-(3,4-dimethoxyphenyl)-1-methyloctahydro-1H-indol-6-yl]-N-methylthiourea trifluoroacetate

Mesembrine (250 mg, 870 μmol) was dissolved in DCM (4 mL). An aqueous solution of methylamine (12 mL of a 50% solution) was added, followed by sodium cyanoborohydride (250 mg, 3.98 mmol). The mixture was stirred overnight at room temperature and the solvent removed under reduced pressure. The crude product was partitioned between NaOH solution (25 mL, 3M) and DCM (25 mL). The aqueous portion was extracted with further DCM (2×20 mL), the combined extracts dried (Na₂SO₄), and the solvent was removed under reduced pressure. The oily residue was dissolved in DCM (5 mL), and treated with n-butylisothiocyanate (115 μL, 110 mg, 960 μmol). After stirring at room temperature for 16 hours, the solvent was removed and the crude products purified by preparative HPLC.
N′-butyl-N-[(3aS*,6R*,7aS*)-3a-(3,4-dimethoxyphenyl)-1-methyloctahydro-1H-indol-6-yl]-N-methylthiourea (29.6 mg, 8%):

MS (ESI+) for C₂₃H₃₇N₃O₂S: m/z 420.3 (M+1). HRMS (EI) calcd C₂₃H₃₇N₃O₂S: 419.2606, found 419.2605
N′-butyl-N-[(3aS*,6S*,7aS*)-3a-(3,4-dimethoxyphenyl)-1-methyloctahydro-1H-indol-6-yl]-N-methylthiourea (40.4 mg, 11%): MS (ESI+) for C₂₃H₃₇N₃O₂S: m/z 420.3 (M+1).

HRMS (EI) calcd C₂₃H₃₇N₃O₂S: 419.2606, found 419.2592

### EXAMPLE 72

### N′-benzyl-N-[(3aS*,6S*,7aS*)-3a-(3,4-dimethoxyphenyl)-1-methyloctahydro-1H-indol-6-yl]-N-methylthiourea and

### EXAMPLE 73

### N′-benzyl-N-[(3aS*,6S*,7aS*)-3a-(3,4-dimethoxyphenyl)-1-methyloctahydro-1H-indol-6-yl]-N-methylthiourea trifluoroacetate

Compounds were prepared and purified in an analogous method to Example 70 and 71.
N′-benzyl-N-[(3aS*,6R*,7aS*)-3a-(3,4-dimethoxyphenyl)-1-methyloctahydro-1H-indol-6-yl]-N-methylthiourea (56.3 mg, 14%): MS (ESI+) for C₂₆H₃₅N₃O₂S: m/z 454.2 (M+1).

HRMS (EI) calcd C₂₆H₃₅N₃O₂S: 453.245, found 454.2442
N′-benzyl-N-[(3aS*,6S*,7aS*)-3a-(3,4-dimethoxyphenyl)-1-methyloctahydro-1H-indol-6-yl]-N-methylthiourea (72.3 mg, 18%): MS (ESI+) for C₂₆H₃₅N₃O₂S: m/z 454.2 (M+1).

HRMS (EI) calcd C₂₆H₃₅N₃O₂S: 453.245, found 454.2444.

### EXAMPLE 74

### N-[(3aS*,6S*,7aS*)-1-benzyl-3a-(3,4-dimethoxyphenyl)octahydro-1H-indol-6-yl]-4-methylpiperazine-1-carboxamide bis(trifluoroacetate)

Triethylamine (0.29 mL, 2.07 mmol) was added to a solution of (3aS*,6S*,7aS*)-1-benzyl-3a-(3,4-dimethoxyphenyl)octahydro-1H-indol-6-amine (Example 6, 0.38 g, 1.04 mmol) in dry CH₂Cl₂ (20 mL). Triphosgene (0.123 g, 0.415 mmol), dissolved in dry CH₂Cl₂ (3 mL), was added to the reaction mixture dropwise. The mixture was stirred at room temperature under N₂ atmosphere for about 30 minutes. During this time the colour changed from light yellow to darker yellow. Volatiles were evaporated which gave the crude isocyanate as a yellow solid. MS (ESI+) m/z 393 (M+H)⁺.

1-Methyl-piperazine (0.164 mL, 1.48 mmol) was added to the crude isocyanate (0.194 g, 0.494 mmol) dissolved in dry CH₂Cl₂ (10 mL), and the mixture was stirred under N₂ atmosphere for 3 hours. Volatiles were evaporated and the crude product was purified by preparative HPLC which gave 0.64 mg of the title compound as an off-white solid. MS (ESI+) m/z 493 (M+H)⁺. HRMS (EI) calc for C₂₉H₄O₃N₄O₃: 492.3100 found 492.3076.

### EXAMPLE 75

### N-[(3 aS*,6R*,7aS*)-3a-(3,4-dimethoxyphenyl)-1-methyloctahydro-1H-indol-6-yl]-4-methylpiperazine-1-carboxamide bis(trifluoroacetate)

Triethylamine (62 μL, 0.448 mmol) was added to a solution of (3aS*,6R*,7aS*)-3a-(3,4-dimethoxyphenyl)-1-methyloctahydro-1H-indol-6-amine (Comparative Example 7, 65 mg, 0.224 mmol) dissolved in dry CH₂Cl₂ (3 mL). Triphosgene (26.6 mg, 0.0895 mmol) was dissolved in dry CH₂Cl₂ (1 mL) and added dropwise. The solution was stirred under N₂ in room temperature for 3 h. 1-Methyl-piperazine (25 μL, 0.224 mmol) was added and the reaction mixture was stirred at room temperature overnight. Volatiles was evaporated and the crude product was purified by preparative HPLC which gave 93 mg (99%) of the title compound. ¹H NMR (400 MHz, MeOH-D4) δ ppm 1.18 (s, 3H), 1.21 (s, 3H), 1.67-1.75 (m, 2H), 2.11 (br s, 2H), 2.26-2.30 (br s, 3H), 2.77-3.35 (m, 8H), 3.69 (s, 3H), 3.73 (s, 3H), 3.78 (br m, 2H), 3.99 (br m, 3H), 6.86 (m, 3H).

MS (ESI+) m/z 417 (M+H)⁺. HRMS (EI) calc for C₂₃H₃₆N₄O₃: 416.2787 found 416.2791.

### EXAMPLE 76

### N-[(3aS*,6R*,7aS*)-3a-(3,4-dimethoxyphenyl)-1-methyloctahydro-1H-indol-6-yl]piperazine-1-carboxamide bis(trifluoroacetate)

Piperazine (9.5 mg, 0.1097 mmol) was added to the crude solution of isocyanate (0.1097 mmol) prepared in comparative example 77. The mixture was stirred at room temperature under N₂ atmosphere overnight. Volatiles were evaporated and the crude product was purified by preparative HPLC which gave 5.8 mg (13%) of Example 76.

MS (ESI+) m/z 403 (M+H)⁺. HRMS (EI) calc for C₂₂H₃₄N₄O₃: 402.2631 found 402.2630. See also under Comparative Example 77.

### COMPARATIVE EXAMPLE 77

### Preparation of Isocyanate for Small Set Library: (3aS*,6R*,7aS*)-3a-(3,4-dimethoxyphenyl)-6-isocyanato-1-methyloctahydro-1H-indole

Triethylamine (305 μL, 2.19 mmol) was added to a solution of (3aS*,6R*,7aS*)-3a-(3,4-dimethoxyphenyl)-1-methyloctahydro-1H-indol-6-amine (Comparative Example 7, 318 mg, 1.097 mmol) dissolved in dry CH₂Cl₂ (5 mL). Triphosgene (130 mg, 0.44 mmol) was dissolved in dry CH₂Cl₂ (1 mL) and added dropwise. The solution was stirred under N₂ in room temperature for 3 h. MS (ESI+) m/z 393 (M+H)⁺. The crude isocyanate was partitioned into several reaction vials, to which the appropriate amine (see below) was added).

### EXAMPLE 78

### N-[(3aS*,6R*,7aS*)-3a-(3,4-dimethoxyphenyl)-1-methyloctahydro-1H-indol-6-yl]-N′-(2-piperazin-1-ylethyl)urea tris(trifluoroacetate)

N-(2-aminoethyl)piperazine (14 μL, 0.1097 mmol) was added to the isocyanate (0.1097 mmol) solution of Comparative Example 77. The mixture was stirred at room temperature under N₂ atmosphere overnight. Volatiles were evaporated and the crude product was purified by preparative HPLC, which gave 39 mg (79%) of the title compound.

MS (ESI+) m/z 446 (M+H)⁺. HRMS (EI) calc for C₂₄H₃₉N₅O₃: 445.3053 found 445.3058.

### EXAMPLE 79

### N-[(3aS*,6R*,7aS*)-3a-(3,4-dimethoxyphenyl)-1-methyloctahydro-1H-indol-6-yl]-N′-[3-(4-methylpiperazin-1-yl)propyl]urea tris(trifluoroacetate)

1-(3-aminopropyl)-4-methylpiperazine (17 mg, 0.1097 mmol) was added to the isocyanate (0.1097 mmol) solution of Comparative Example 77. The mixture was stirred at room temperature under N₂ atmosphere overnight. Volatiles were evaporated and the crude product was purified by preparative HPLC, which gave 22 mg (42%) of the title compound. MS (ESI+) m/z 474 (M+H)⁺. HRMS (EI) calc for C₂₆H₄₃N₅O₃: 473.3366 found 473.3364.

### EXAMPLE 80

### N-[(3aS*,6R*,7aS*)-3a-(3,4-dimethoxyphenyl)-1-methyloctahydro-1H-indol-6-yl]-N′-[(5-methylpyrazin-2-yl)methyl]urea trifluoroacetate

2-(Aminomethyl)-5-methylpyrazine (21 mg, 0.17 mmol) was dissolved in 1 mL dry CH₂Cl₂ under N₂. Diisopropylamine (44 mg, 0.34 mmol was added followed by dropwise addition of triphosgene (24 mg, 0.08 mmol) in 1 mL of dry CH₂Cl₂. Stirred at room temperature for 2 hrs, and then (3aS*,6R*,7aS*)-3a-(3,4-dimethoxyphenyl)-1-methyloctahydro-1H-indol-6-amine (Comparative Example 7, 50 mg, 0.17 mmol) was added. Stirred at room temperature overnight and then concentrated. Purification using preparative HPLC gave the product as light yellow oil (7.8 mg, 10%).

¹HNMR (270 MHz, Chloroform-d) ppm 1.04-1.32(m, 5H); 1.76-1.90(m, 3H); 2.00-2.38(m, 5H); 2.53(s, 3H); 3.46-3.72(m, 1H); 3.87(s, 6H); 4.05-4.20(m, 1H); 4.48(s, 2H); 5.21(w, 1H); 6.70-6.98(m, 2H); 7.30-7.65(m, 1H); 8.36-8.40(m, 2H); 8.47(b,1H).

MS (ESI⁺) for C₂₄H₃₃N₅O₃ m/z 440 (M+H⁺), HRMS found: 439,2580 calculated: 439,2583

### EXAMPLE 81

### N-[(3aS*,6R*,7aS*)-3a-(3,4-dimethoxyphenyl)-1-methyloctahydro-1H-indol-6-yl]-N′-(4-methylipiperazin-1-yl)urea tris(trifluoroacetate)

1-Amino-4-methylpiperazine (13 μL, 0.1097 mmol) was added to the isocyanate (0.1097 mmol) solution of Comparative Example 77. The mixture was stirred at room temperature under N₂ atmosphere overnight. Volatiles were evaporated and the crude product was purified by preparative HPLC, which gave 33 mg (69%) of the title compound.

MS (ESI+) m/z 432 (M+H)⁺. HRMS (EI) calc for C₂₃H₃₇N₅O₃: 431.2896 found 431.2875.

### EXAMPLE 82

### N-[(3aS*,6R*,7aS*)-1-benzyl-3a-(3,4-dimethoxyphenyl)octahydro-1H-indol-6-yl]-N′-cyclopropylthiourea trifluoroacetate

Reagent: isothiocyanatocyclopropane

Synthetic procedure: Scheme A

Yield: 2.2 mg

Measured mass: 465.2468

Calc. mass: 465.2450

### EXAMPLE 83

### N-[(3aS*,6R*,7aS*)-1-benzyl-3a-(3,4-dimethoxyphenyl)octahydro-1H-indol-6-yl]-N′-(sec-butyl)thiourea trifluoroacetate

Reagent: 2-isothiocyanatobutane

Synthetic procedure: Scheme A

Yield: 15.5 mg

Measured mass: 481.2767

Calc. mass: 481.2763

### EXAMPLE 84

### N-[(3aS*,6R*,7aS*)-1-benzyl-3a-(3,4-dimethoxyphenyl)octahydro-1H-indol-6-yl]-N′-cyclopentylthiourea trifluoroacetate

Reagent: cyclopentyl isothiocyanate

Synthetic procedure: Scheme A

Yield: 15.4 mg

Measured mass: 493.2786

Calc. mass: 493.2763

### EXAMPLE 85

### N-[(3aS*,6R*,7aS*)-3a-(3,4-dimethoxyphenyl)-1-methyloctahydro-1H-indol-6-yl]-N′-[(1S)-1-phenylethyl]urea trifluoroacetate

Reagent: (S)-(−)-alpha-methylbenzyl isocyanate

Synthetic procedure: Scheme C

Yield: 2.3 mg

Measured mass: 437.2691

Calc. mass: 437.2678

### EXAMPLE 86

### N-allyl-N′-[(3aS*,6R*,7aS*)-3a-(1,3-benzodioxol-5-yl)-1-benzyloctahydro-1H-indol-6-yl]urea hydrochloride and

### EXAMPLE 87

### N-allyl-N′-6(3aS*,6S*7aS*)-3a-(1,3-benzodioxol-5-yl)-1-benzyloctahydro-1H-indol-6-yl]urea hydrochloride

LiNH₂ (7.5 g, 328 mmol) was suspended in DME (200 mL) at ambient temperature and (3,4-methylenedioxy)phenylacetonitrile (20 g, 124 mmol) in DME (50 mL) was added portionwise over 15 min. The mixture was heated at 80° C. for 30 min, whereupon its color changed to green, before a solution of 1-bromo-2-chloroethane (11.3 mL, 136 mmol) in DME (50 mL) was added over a period of 20 min. During the course of the addition, the green color of the mixture changed to light brown. The mixture was heated at 80° C. overnight, or until GC indicated >95% consumption of the starting material. The mixture was cooled on an ice/water bath, and water (200 mL) and Et₂O (400 ml) was then added to destroy the excess of strong base. The mixture was extracted with DCM (2×100 mL), and the combined organic extracts were washed with H₂O (100 mL), dried (MgSO₄) and evaporated. The residue was purified by flash chromatography (silica, 10-20% EtOAc in n-heptane) to yield 1-(3,4-methylenedioxyphenyl)cyclopropanecarbonitrile (19.0 g, 82%) as a yellowish oil. ¹H NMR (270 MHz, CDCl₃) δ ppm 1.18-1.32 (m, 2H); 1.57-1.66 (m, 2H); 5.93 (s, 2H); 6.71-6.83 (m, 3H).

1-(3,4-methylenedioxyphenyl)cyclopropanecarbonitrile (19.0 g of crude material, 101 mmol), was dissolved in dry toluene (800 mL) and cooled on an ice/water bath. A solution of DIBAL (1M in toluene, 140 mL, 140 mmol) was added dropwise via an addition funnel, over a period of 30 min. The resulting mixture was heated at 50° C. overnight. The reaction mixture was cooled to 0° C. and cautiously transferred, in small portions and with swirling, to a separatory funnel containing ice-cold aq. HCl (4M, 0.5 L). The aqueous layer was extracted once with EtOAc (400 mL) and the combined organic portions were washed with water (1×300 mL) and brine (1×200 mL), dried (MgSO₄) and concentrated to give the aldehyde (19 g) as a yellowish oil, which was used in the subsequent step without further purification. ¹H NMR (270 MHz, CDCl₃) δ 1.32-1.37 (m, 2H); 1.49-1.55 (m, 2H); 5.95 (s, 2H); 6.69-6.83 (m, 3H); 9.18 (s, 1H).

To a solution of the aldehyde (19 g of crude material, assumed to be 101 mmol) in dry THF (800 mL) was added benzylamine (11.9 g, 111 mmol) and an excess of MgSO₄ (50 g) and the resulting mixture was stirred at rt during 24 h. The mixture was filtered and evaporated to give the imine (28 g), which was used in the next step without further purification. ¹H NMR (270 MHz, CDCl₃) δ 1.12-1.17 (m, 2H); 1.30-1.36 (m, 2H); 4.56 (s, 2H); 5.93 (s, 2H); 6.69-6.89 (m, 3H); 7.14-7.40 (m, 5H); 7.70 (s, 11H).

To a solution of imine (10 g of crude material, assumed to be 53 mmol) and benzylamine hydrochloride (10 g, 68 mmol) in MeCN (400 mL) was added Na₂SO₄ (20 g) and but-3-en-2-one (3.7 g, 53 mmol). The mixture was heated at reflux for 5 h and then cooled to rt. The drying agent was filtered off and the filtrate was evaporated to dryness. The residue was partitioned between EtOAc (200 mL) and saturated aqueous NaHCO₃ (100 mL), and the aqueous layer was extracted with EtOAc (2×100 mL). The combined organic portions were washed with brine (100 mL), dried (MgSO₄) and concentrated to give (3aS*,7aS*)-3a-benzo[1,3]dioxol-5-yl-1-benzyl-octahydroindol-6-one as a colorless oil (29%) after purification by column chromatography (silica/hexanes: EtOAc 70:30).

¹H NMR (270 MHz, CDCl₃) δ 1.85-2.36 (m, 6H); 2.40-2.82 (m, 3H); 2.87-2.98 (m, 1H); 3.10 (d, 11H, J=12.1 Hz); 3.21-3.26 (m, 11H); 4.08 (d, 11H, J=12.1 Hz); 5.93 (s, 2H); 6.71-6.95 (m, 3H); 7.17-7.41 (m, 5H). ¹³C NMR (67.9 MHz, CDCl₃) 34.86, 36.08, 38.45, 40.36, 47.24, 51.46, 57.30, 68.24, 100.88, 106.73, 107.86, 118.48, 126.75, 128.02, 128.57, 138.78, 141.52, 145.63, 147.86, 210.96.

To a solution of (3aS*,7aS*)-3a-benzo[1,3]dioxol-5-yl-1-benzyl-octahydroindol-6-one (1.5 g, 4.30 mmol) and ammonium formate (2 g, 38 mmol) in MeOH (100 mL) was added NaBH₃CN (2 g, 32 mmol) in portions during 5 min. The resulting mixture was stirred at rt for 4 h and then evaporated. The residue was partitioned between EtOAc (100 mL) and saturated aqueous NaHCO₃ (50 mL). The aqueous phase was extracted with EtOAc (2×50 mL) and the combined organic fractions were washed with brine (50 mL), dried (MgSO₄) and evaporated to give (3aS*,7aS*)-3a-benzo[1,3]dioxol-5-yl-1-benzyl-octahydro-indol-6-ylamine as a colorless oil (1.3 g, 87%), which was used in the next step without further purification. ¹H NMR indicated the formation of approximately a 1:1 mixture of diastereomers (at C-6).

¹H NMR (270 MHz, CDCl₃) δ 0.89-2.42 (m, 11H); 2.83-3.30 (m, 3H); 4.18 (d, 0.5H, J=13.7 Hz); 4.30 (d, 0.5H, J=13.0 Hz); 5.88 (s, 2H); 6.64-7.00 (m, 3H); 7.10-7.53 (m, 5H).

The general procedure for urea/thiourea formation was used, starting from (3aS*,7aS*)-3a-benzo[1,3]dioxol-5-yl-1-benzyl-octahydro-indol-6-ylamine (0.1 g, 0.29 mmol) and allyl isocyanate (34 mg, 0.4 mmol), to give 102 mg (74%) of the mixture of isomers. The isomers were separated by using column chromatography (silica, CHCl₃/MeOH/NH₃) and converted to the corresponding hydrochlorides via treatment of DCM solutions with HCl/Et₂O (sat.) followed by evaporation. First eluted: N-allyl-N′-[(3aS*,6R*,7aS*)-3a-(1,3-benzodioxol-5-yl)-1-benzyloctahydro-1H-indol-6-yl]urea hydrochloride: 23 mg (17%). ¹H NMR (500 MHz, CDCl₃): δ ppm 1.16-1.34 (m, 1H); 1.55-1.68 (m, 1H); 1.82 (d, 1H, J=14.1 Hz); 1.91-2.26 (m, 4H); 3.21-3.31 (m, 1H); 3.73-4.08 (m, 4H); 4.21 (dd, 1H, J=13.0, 4.9 Hz); 4.45-4.54 (m, 1H); 4.56 (d, 1H, J=13.2 Hz); 5.11 (d, 1H, J=10.6 Hz); 5.21 (dd, 1H, J=16.7, 4.8 Hz); 5.44-5.64 (br s, 1H); 5.82-5.95 (m, 1H, J=16.7, 10.6, 5.0 Hz); 5.97 (s, 2H); 6.59 (d, 1H, J=8.46 Hz); 6.65 (s, 1H); 6.78 (d, 1H, J=8.46 Hz); 6.68-7.05 (br s, 1H); 7.45-7.53 (m, 3H); 7.65 (d, 2H, J=7.06 Hz); 11.77-12.26 (br s, 1H).

MS (ESI+): m/z 434 (M+1). HRMS (EI) Calc for C₂₆H₃₁N₃O₃: 433.2365; found 433.2553.

Second eluted: N-allyl-N′-[(3aS*,6S*,7aS*)-3a-(1,3-benzodioxol-5-yl)-1-benzyloctahydro-1H-indol-6-yl]urea hydrochloride:greyish gummy solid; 49 mg (36%). The relative configuration was determined by ¹H NMR spectroscopy (NOESY). ¹H NMR (270 MHz, CDCl₃): δ ppm 1.23-1.47 (m, 2H); 1.54-1.70 (m, 1H); 1.77-1.96 (m, 2H); 1.98-2.34 (m, 2H); 2.35-2.38 (m, 1H); 3.04-3.17 (m, 1H); 3.20-3.35 (m, 2H), 4.21 (d, 1H, J=13.0 Hz); 4.35-4.50 (m, 1H); 4.63-4.80 (m, 1H); 4.94-5.23 (m, 2H); 5.65-5.88 (m, 1H); 5.93 (s, 2H); 6.67-6.87 (m, 3H); 7.23-7.37 (m, 5H). MS (ESI+): m/z 434 (M+1). HRMS (EI) Calc for C₂₆H₃₁N₃O₃: 433.2365; found 433.2358.

### EXAMPLE 88

### N-[(3aS*,6R*,7aS*)-3a-(1,3-benzodioxol-5-yl)-1-benzyloctahydro-1H-indol-6-yl]-N′-ethylthiourea hydrochloride and

### EXAMPLE 89

### N-[(3aS*,6S*,7aS*)-3a-(1,3-benzodioxol-5-yl)-1-benzyloctahydro-1H-indol-6-yl]-N′-ethylthiourea hydrochloride

The general procedure for urea/thiourea formation was used, starting from from (3aS*,7aS*)-3a-benzo[1,3]dioxol-5-yl-1-benzyl-octahydro-indol-6-ylamine, intermediate from Example 86 and 87 (0.1 g, 0.29 mmol) and ethyl isothiocyanate (36 mg, 0.4 mmol), to give 111 mg (81%) of the mixture of isomers. The isomers were separated by using column chromatography (silica, CHCl₃/MeOH/NH₃) and converted to the corresponding hydrochlorides via treatment of DCM solutions with HCl/Et₂O (sat.) followed by evaporation. N-[(3aS*,6R*,7aS*)-3a-(1,3-benzodioxol-5-yl)-1-benzyloctahydro-1H-indol-6-yl]-N′-ethylthiourea hydrochloride (slower elute): greyish gummy solid; 30 mg (20%). The relative configuration was determined by ¹H NMR spectroscopy (NOESY).

¹H NMR (270 MHz, CDCl₃): δ ppm 0.81-0.96 (m, 1H); 1.03 (t, 3H, J=7.05 Hz); 1.10-1.52 (m, 2H); 1.55-1.75 (m, 2H); 1.75-2.10 (m, 5H); 2.18 (d, 1H, J=15.6 Hz); 2.56-2.71 (m, 1H); 2.87-3.54 (m, 3H); 4.03-4.50 (m, 1H); 5.12-5.26 (br s, 1H); 5.94 (s, 2H); 6.69-6.89 (m, 3H); 7.23-7.44 (m, 5H). MS (ESI+): m/z 438 (M+1). HRMS (EI) Calc for C₂₅H₃₁N₃O₂S: 437.2137; found 437.2151.

Faster elute: N-[(3aS*,6S*,7aS*)-3a-(1,3-benzodioxol-5-yl)-1-benzyloctahydro-1H-indol-6-yl]-N′-ethylthiourea hydrochloride: greyish gummy solid; 58 mg (40%). The relative configuration was determined by ¹H NMR spectroscopy (NOESY). ¹H NMR (270 MHz, CDCl₃): δ ppm 1.03-1.16 (m, 4H); 1.16-1.32 (m, 3H); 1.36-1.52 (m, 1H); 1.60-1.93 (m, 3H); 1.94-2.06 (m, 1H); 2.10-2.48 (m, 2H); 2.95-3.13 (m, 2H); 3.16-3.38 (m, 2H); 4.15-4.28 (m, 1H); 5.27-5.51 (br s, 1H); 5.94 (s, 2H); 6.69-6.89 (m, 3H); 7.27-7.48 (m, 5H).

MS (ESI+) m/z 438 (M+1). HRMS (EI) Calc for C₂₅H₃₁N₃O₂S: 437.2137; found 437.2124.

### EXAMPLE 90

### N-[(3aS*,6S*,7aS*)-3a-(1,3-benzodioxol-5-yl)-1-benzyloctahydro-1H-indol-6-yl]-N′-benzylthiourea hydrochloride and

The general procedure for urea/thiourea formation was used, starting from (3aS*,7aS*)-3a-benzo[1,3]dioxol-5-yl-1-benzyl-octahydro-indol-6-ylamine, intermediate from Example 86 and 87 (0.9 g, 2.57 mmol) and benzyl isothiocyanate (500 mg, 3.3 mmol), to give 960 mg (65%) of the mixture of isomers. The isomers were separated by using column chromatography (silica, CHCl₃/MeOH/NH₃) and converted to the corresponding hydrochlorides via treatment of DCM solutions with HCl/Et₂O (sat.) followed by evaporation. N-[(3aS*,6S*,7aS*)-3a-(1,3-benzodioxol-5-yl)-1-benzyloctahydro-1H-indol-6-yl]-N′-benzylthiourea hydrochloride (faster eluting): colorless gummy solid; 500 mg (17%). The relative configuration was determined by ¹H NMR spectroscopy (NOESY).

¹H NMR (270 MHz, CDCl₃) δ ppm 0.81-0.96 (m, 1H); 1.16-1.47 (m, 2H); 1.53-2.48 (m, 10H); 2.92-3.26 (m, 2H); 4.09-4.23 (m, 2H); 4.53-4.63 (br s, 1H); 5.94 (s, 2H); 6.71-6.90 (m, 3H); 7.15-7.42 (m, 10H). MS (ESI+) m/z 500 (M+1). HRMS (EI) Calc for C₃₀H₃₃N₃O₂S: 499.2293; found: 499.2277

### EXAMPLE 91

### N-[(3aS*,6R*,7aS*)-3a-(1,3-benzodioxol-5-yl)-1-benzyloctahydro-1H-indol-6-yl]-N′-butylthiourea hydrochloride

The general procedure for urea/thiourea formation was used, starting from (3aS*,7aS*)-3a-benzo[1,3]dioxol-5-yl-1-benzyl-octahydro-indol-6-ylamine, intermediate from Example 86 and 87 (0.1 g, 0.29 mmol) and n-butyl isothiocyanate (46 mg, 0.4 mmol), to give 95 mg (68%) of the mixture of isomers. The isomers were separated by using column chromatography (silica, CHCl₃/MeOH/NH₃) and converted to the corresponding hydrochlorides via treatment of DCM solutions with HCl/Et₂O (sat.) followed by evaporation. The title compound (slower elute): greyish gummy solid; 48 mg (33%). The relative configuration was determined by ¹H NMR spectroscopy (NOESY).

¹H NMR (270 MHz, CDCl₃) δ ppm 0.86 (t, 3H, J=7.3 Hz); 1.06-2.06 (m, 12H); 2.18 (d, 1H, J=16.0 Hz); 2.55-2.70 (m, 1H); 2.85-3.33 (m, 3H); 3.45 (d, 1H, J=13.3 Hz); 4.04-4.14 (m, 1H); 4.58-4.75 (br s, 1H); 5.19-5.33 (br s, 1H); 5.93 (s, 2H); 6.70-6.87 (m, 3H); 7.23-7.45 (m, 5H) 8.43-8.59 (br s, 1H). MS (ESI+): m/z 466 (M+1). HRMS (EI) Calc for C₂₇H₃₅N₃O₂S: 465.2450; found 465.2435.

### EXAMPLE 92

### N-[(3aS*,6R*,7aS*)-3a-(1,3-benzodioxol-5-yl)-1-benzyloctahydro-1H-indol-6-yl]-N′-(tert-butyl)thiourea hydrochloride

The general procedure for urea/thiourea formation was used, starting from (3aS*,7aS*)-3a-benzo[1,3]dioxol-5-yl-1-benzyl-octahydro-indol-6-ylamine, intermediate from Example 86 and 87 (0.1 g, 0.29 mmol) and t-butyl isothiocyanate (46 mg, 0.4 mmol), to give 103 mg (74%) of the mixture of isomers. The isomers were separated by using column chromatography (silica, CHCl₃/MeOH/NH₃) and converted to the corresponding hydrochlorides via treatment of DCM solutions with HCl/Et₂O (sat.) followed by evaporation. The title compound (slower elute): colorless gummy solid; 19 mg (14%). The relative configuration was determined by ¹H NMR spectroscopy (NOESY).

¹H NMR (270 MHz, CDCl₃) δ ppm 0.80-0.97 (m, 1H); 1.16-1.47 (m, 11H); 1.62-2.32 (m, 7H); 2.57 (d, 11H, J=14.0 Hz); 2.88-3.20 (m, 2H); 4.59-4.80 (br s, 1H); 5.29 (d, 1H, J=9.0 Hz); 5.78-5.90 (br s, 1H); 5.93 (s, 2H); 6.72-6.91 (m, 3H); 7.16-7.49 (m, 5H).

MS (ESI+): m/z 466 (M+1). HRMS (EI) Calc for C₂₇H₃₅N₃O₂S: 465.2450; found 465.2448.

### EXAMPLE 93

### N-[(3aS*,6S*,7aS*)-3a-(1,3-benzodioxol-5-yl)-1-benzyloctahydro-1H-indol-6-yl]-N′-(tert-butyl)thiourea hydrochloride

The general procedure for urea/thiourea formation was used, starting from (3aS*,7aS*)-3a-benzo[1,3]dioxol-5-yl-1-benzyl-octahydro-indol-6-ylamine, intermediate from Example 86 and 87 (0.1 g, 0.29 mmol) and t-butyl isothiocyanate (46 mg, 0.4 mmol), to give 103 mg (74%) of the mixture of isomers. The isomers were separated by using column chromatography (silica, CHCl₃/MeOH/NH₃) and converted to the corresponding hydrochlorides via treatment of DCM solutions with HCl/Et₂O (sat.) followed by evaporation. Example 91 (faster elute): colorless gummy solid; 48 mg (34%). The relative configuration was determined by ¹H NMR spectroscopy (NOESY).

¹H NMR (270 MHz, CDCl₃) δ ppm 1.20-1.32 (m, 1H); 1.42 (s, 9H); 1.58-1.73 (m, 1H); 1.49-2.05 (m, 6H); 2.24 (d, 1H, J=16.5 Hz); 2.62-2.79 (m, 1H); 3.10-3.23 (m, 2H); 3.70 (d, 1H, J=13.8 Hz); 4.00 (d, 11H, J=13.8 Hz); 5.55-5.65 (br s, 1H); 5.93 (s, 2H); 6.68-6.80 (m, 3H); 7.25-7.40 (m, 5H); 7.94-8.11 (br s, 1H). MS (ESI+): m/z 466 (M+1). HRMS (EI) Calc for C₂₇H₃₅N₃O₂S: 465.2450; found 465.2450.

### EXAMPLE 94

### N-[(3aS*,6S*,7aS*)-3a-(1,3-benzodioxol-5-yl)-1-benzyloctahydro-1H-indol-6-yl]-N′-butylthiourea hydrochloride

The general procedure for urea/thiourea formation was used, starting from (3aS*,7aS*)-3a-benzo[1,3]dioxol-5-yl-1-benzyl-octahydro-indol-6-ylamine, intermediate from Example 86 and 87 (0.1 g, 0.29 mmol) and n-butyl isothiocyanate (46 mg, 0.4 mmol), to give 95 mg (68%) of the mixture of isomers. The isomers were separated by using column chromatography (silica, CHCl₃/MeOH/NH₃) and converted to the corresponding hydrochlorides via treatment of DCM solutions with HCl/Et₂O (sat.) followed by evaporation. The title compound (faster eluting): colorless gummy solid; 30 mg (21%). The relative configuration was determined by ¹H NMR spectroscopy (NOESY).

¹H NMR (270 MHz, CDCl₃) δ ppm 0.82-0.96 (m, 4H); 1.14-2.52 (m, 14H); 2.96-3.36 (m, 4H); 4.20-4.31 (m, 2H); 5.32-5.61 (br s, 1H); 5.93 (s, 2H); 6.71-6.90 (m, 3H); 7.24-7.50 (m, 5H). MS (ESI+) m/z 466 (M+1). HRMS (EI) Calc for C₂₇H₃₅N₃O₂S: 465.2450; found 465.2451.

### EXAMPLE 95

### N-[(3aS*,6R*,7aS*)-3a-(1, 3-benzodioxol-5-yl)-1-benzyloctahydro-1H-indol-6-yl]-N′-benzylthiourea hydrochloride

The general procedure for urea/thiourea was used, starting from (3aS*,7aS*)-3a-benzo[1,3]dioxol-5-yl-1-benzyl-octahydro-indol-6-ylamine, intermediate from Example 86 and 87 (0.9 g, 2.57 mmol) and benzyl isothiocyanate (500 mg, 3.3 mmol), to give 960 mg (71%) of a mixture of isomers. The isomers were separated by using column chromatography (silica, CHCl₃/MeOH/NH₃) and converted to the corresponding hydrochlorides via treatment of DCM solutions with HCl/Et₂O (sat.) followed by evaporation. The title compound (slower elute): colorless gummy solid; 350 mg (12%). The relative configuration was determined by ¹H NMR spectroscopy (NOESY). ¹H NMR (270 MHz, CDCl₃) δ ppm 0.79-0.93 (m, 1H); 1.14-2.60 (m, 8H); 3.00-3.15 (m, 1H); 3.38 (1H, d, J=13.3 Hz); 3.95-4.78 (m, 4H); 4.64-4.75 (br s, 1H); 5.53-5.56 (br s, 1H); 5.94 (s, 2H); 6.68-6.86 (m, 3H); 7.16-7.41 (m, 10H). MS (ESI+) m/z 500 (M+1). HRMS (EI) Calc for C₃₀H₃₃N₃O₂S: 499.2293; found: 499.2283

Preparation of a Pharmaceutical Composition

### EXAMPLE 96

### Preparation of Tablets

| Ingredients | mg/tablet |
|---|---|
| 1. Active compound of formula (I) | 10.0 |
| 2. Cellulose, microcrystalline | 57.0 |
| 3. Calcium hydrogen phosphate | 15.0 |
| 4. Sodium starch glycolate | 5.0 |
| 5. Silicon dioxide, colloidal | 0.25 |
| 6. Magnesium stearate | 0.75 |

The active ingredient 1 is mixed with ingredients 2, 3, 4 and 5 for about 10 minutes. The magnesium stearate is then added, and the resultant mixture is mixed for about 5 minutes and compressed into tablet form with or without film-coating.

Biological Methods

The ability of a compound of the invention to bind or act at the MCH1R receptor can be determined using in vitro and in vivo assays known in the art. The biological activity of compounds prepared in the Examples was tested using different tests.

Binding Assay

The compounds according to the invention were evaluated for their binding to the human MCH1R receptor by the following method:

Materials and Methods

Materials

Compounds: MCH peptide was purchased from Phoenix pharmaceuticals. (Phe¹³, [¹²⁵I]Tyr¹⁹ Melanine-Concentrating Hormone (human, mouse, rat) ([¹²⁵I]-MCH) was obtained from NEN life Science Products. Inc. Boston, Mass. Wheat germ agglutinine SPA beads (RPNQ 0001) were obtained from Amersham-Pharmacia Biotech. All other reagents used are of highest purity from different resources available. Protein Kits, Micro BCA™ Protein Assay Reagent Kit (Cat No. 23235) were purchased from Piece, Rockford, Ill., USA.

Plastic wares: Cell culture flasks, dishes were from Decton Dickinson Labware, N.J., USA. Scintillation plate, white clear bottom were from Wallac, Finland.

Cells and Culture Conditions

CHO-K1 cells expressing hMCH1 receptor were purchased from Euroscreen. CHO-K1 hMCHRI (Euroscreen, Brussels, Belgium, # ES-370-C) were cultivated in Nutrient mixture Ham's F-12 with Glutamax I (Gibco-BRL #31765-027) supplemented with 10% heat-inactivated foetal calf serum (FCS, Gibco-BRL #10108-165) and 400 μg/ml geniticin (Gibco-BRL #1140-0359). The cells were sub-cultivated twice weekly with split ratio=1:20-1:30. For membrane preparation the cells were cultured in 500 mm² dishes and the cells were harvested when 90% confluent.

Membrane Preparation

When the cells reached more than 90% confluence, dishes (500 cm²) were rinsed twice with 20 ml PBS (Ca²⁺ and Mg²⁺ free). Buffer A, which contains Tris.HCl (15), MgCl₂.6H₂O (2), EDTA (0.3), EGTA (1) in mM with pH 7.5, 25 ml was added and cells were suspended using a window scraper. The cells were collected in 50 ml Falcon tube pre-cooled on ice and then centrifuged for 3 minutes at 1500 g at 4° C. The supernatant was discarded and the cells were suspended again with Buffer A. The cells were homogenized using a Polytron homogenizer at setting 4 for 4 times for 30 seconds with 1 minute pause between the cycles. The homogenized preparation was centrifuged at 40,000 g (18500 rpm with ss-34, No. 5 rotor in Sorvall centrifuge, RC5C, DuPont) for 25 minutes at 4° C. The pellets were washed once with Buffer A and centrifuged again under the same conditions. The pellets were suspended with Buffer B, which contains Tris.HCl (7.5), MgCl₂.6H₂O (12.5), EDTA (0.3), EGTA (1), sucrose (25) in mM with pH 7.5, and gently homogenized for several times with a glass homogenizer. The membrane preparation was aliquoted into Eppendorf tubes, 1 ml/tube and frozen at −70° C.

Membrane Protein Determination

The protein determination was done as described in the instruction provided with Pierce protein assay kit (Peirce Micro BCA Protein assay reagent kit, No 23235, Pierce, USA). Briefly, the Piece working reagent components A, B and C were mixed in the ratio 25:24:1. BSA (No. 23209, Pierce, USA) provided with the kits was used as standard, which the concentration in the curve is 1, 2, 4, 6, 8, 12, 16 and 24 μl/ml. The samples from membrane preparation were diluted for 50, 100, 200, 400 times. The standards or the samples 150 μl and the working reagent 150 μl were mixed in each well in a Costa 96 well microtiter plate and incubated at 37° C. for 2 hours. The plate was cooled down to room temperature and read at 595 nm with a Microplate reader from Molecular Devices, USA.

Receptor Binding by SPA

The WGA beads were re-constructed with reaction buffer, which contains Tris (50), MgCl₂ (5), EDTA (2.5) in mM with pH adjusted to 7.4, to 40 mg/ml as a stock suspension. To link the membrane with the bead, the beads and the membrane will be pre-incubated with for 30 minutes at room temperature with gentle shaking. The suspension of the beads was centrifuged at 3400 rpm for 2 minutes using centrifuge. The supernatant was discarded and the beads were re-suspended with binding buffer, HEPES (25 mM), MgCl₂ (5 mM), CaCl₂ (1 mM), BSA (0.5%) with peptidase inhibitors (1 μg/ml) Leupeptin, Aprotinin and pepstatin, pH 7.4.

Since appropriated beads and membrane construction is needed for SPA, the ratio of beads and membrane in link were tested and it will be indicated where the experiments are described.

The radio labeled [¹²⁵I]-MCH was diluted with cold MCH in ratio 1:3. In Kd determination, the concentrations of labeled peptide were 3 nM with 1:2 series dilution for 11 samples. The amount of the beads was 0.25 mg/well. The results were calculated using Excel program and the curves were drawn using a program GraphPad Prism.

For screening of the substances the amount of the beads used was 0.25 mg/well and the amount of the membrane protein was 4 μg/well 0.2 nM of labeled MCH was used. The total volume was 200 μl, which contained 50 μl [¹²⁵I]-MCH, 100 μl substances and 50 μl beads. The plate was gently shaken for 30 minute and incubated overnight. The samples were counted using Microbeta counter (Wallac Trilux 1450 Micro beta counter, Wallac, Finland) for 2 minutes and the results were calculated by using the computer program Activity Base.

Results

The equilibrium time of the binding was investigated at room temperature, 30 and 37° C. The equilibrium time was about 30 minutes at 37° C. but the binding was lower compared with that at room temperature and 30° C. The equilibrium time was about 2 hours at 30° C. while it took about 4 hours to reach stable binding at room temperature. Thus, room temperature was chosen since it is easy condition for experiments.

The [¹²⁵I]-MCH binding to hMCH R1 was further characterized by determination of Kd values. The Kd values are same, 0.19 nM, as reported by Chambers J, Ames R S, Bergsma D, Muir A, Fitzgerald L R, Hervieu G, Dytko G M, Foley J J, Martin J, Liu W S, Park J, Ellis C, Ganguly S, Konchar S, Cluderay J, Leslie R, Wilson S, Sarau H M. Melanin-concentrating hormone is the cognate ligand for the orphan G-protein-coupled receptor SLC-1. Nature 1999 Jul. 15;400(6741):261-5.

In all displacement experiments, 0.2 nM [¹²⁵I]-MCH was used for total binding and 300 nM MCH used as non-specific binding. The background is low and the signal is good. The Z′ factor was 0.83 which is considered very good for screening.

Kd values from present study were consistent with that from Macdonald D, Murgolo N, Zhang R, Durkin J P, Yao X, Strader C D, Graziano M P. Molecular characterization of the melanin-concentrating hormone/receptor complex: identification of critical residues involved in binding and activation. Mol Pharmacol 2000 July;58(1):217-25 but were slightly different from that 1.2 nM from Hervieu G J, Cluderay J E, Harrison D, Meakin J, Maycox P, Nasir S, Leslie R A, The distribution of the mRNA and protein products of the melanin-concentrating hormone (MCH) receptor gene, slc-1, in the central nervous system of the rat. Eur J Neurosci 2000 April; 12(4):1194-216. The reason for this is unknown but might be caused by different clones of the cells.

The calculation of the Kᵢ values for the inhibitors was performed by use of Activity Base. The Kᵢ value is calculated from IC₅₀ and the Kₘ value is calculated using. the Cheng Prushoff equation (with reversible inhibition that follows the Michaelis-Menten equation): Kᵢ=IC₅₀(1+[S]/Kₘ) [Cheng, Y. C.; Prushoff, W. H. Biochem. Pharmacol. 1973, 22, 3099-3108]. The IC₅₀ is measured experimentally in an assay wherein the decrease of the turnover of cortisone to cortisol is dependent on the inhibition potential of each substance.

The compounds of formula (I) exhibit the IC₅₀ values for the MCH1R receptor in the range from 10 nM to 10 μM. Illustrative of the invention, the following Ki values have been determined in the assay (see Table 1):

TABLE 1
Ki values determined in the assay.

| Compound of Example | Ki (nM) |
|---|---|
| 23 | 43 |
| 58 | 907 |
| 69 | 227 |

1. A compound of the general formula (I) 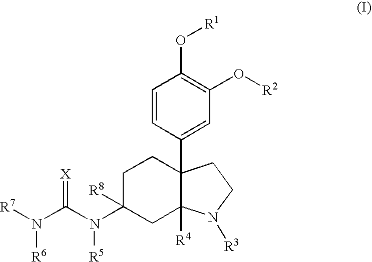
or a pharmaceutically acceptable salt, hydrates, geometrical isomers, racemates, tautomers, optical isomers, N-oxides and prodrug forms thereof, wherein:
R¹ is C₁₋₆-alkyl;
R² is C₁₋₆-alkyl;
or R¹ and R² are linked to form C₁₋₃-alkylene;
R³ is H, C₁₋₆-alkyl, C₁₋₆-alkoxycarbonyl, C₁₋₆-alkoxycarbonyl-C₁₋₆-alkyl, C₁₋₆-alkoxycarbonyl-C₃₋₆-cycloalkyl-C₁₋₆ alkyl, aryl-C₁₋₆-alkyl, heteroaryl-C₁₋₆ alkyl, aryl or heteroaryl, wherein any aryl or heteroaryl may be unsubstituted or independently substituted with C₁₋₆-alkyl, C₁₋₆-alkoxy, C₁₋₆-alkoxycarbonyl, C₁₋₆-alkoxycarbonyl-C₁₋₆-alkyl, C₁₋₆-alkoxycarbonyl-C₃₋₆-cycloalkyl-C₁₋₆ alkyl, halo-C₁₋₆-alkyl, halo-C₁₋₆-alkoxy, or nitro;
R⁴ is H or C₁₋₆-alkyl;
R⁵ is H, OH, C₁₋₆-alkyl, C₁₋₆-alkylaminocarbonyl, or C₁₋₆-alkylaminothioxylcarbonyl;
R⁶ is H or C₁₋₆-alkyl;
R⁷ is H, C₁₋₆-alkyl, C₂₋₆-alkenyl, C₃₋₈-cycloalkyl, C₃₋₈-cycloalkyl-C₁₋₆-alkyl, C₁₋₆-alkoxy, C₁₋₆-alkoxy-C₁₋₆-alkyl, C₁₋₆-alkylcarbonyl, C₁₋₆-alkoxycarbonyl, C₁₋₆-alkoxycarbonyl-C₁₋₆-alkyl, heterocyclo-C₁₋₆-alkyl, heteroaryl-C₁₋₄-alkyl, aryl-C₁₋₆-alkyl, arylcarbonyl, heteroarylcarbonyl, or heterocyclyl, wherein any aryl, heteroaryl and heterocyclo may be unsubstituted or substituted in one, two or three positions with C₁₋₆-alkyl, halo-C₁₋₆-alkyl, C₁₋₆-alkoxy, or halogen;
or R⁶ and R⁷ are linked to form C₄₋₆-alkylene or together with the nitrogen atom to which they are attached form a piperazine ring, which may be unsubstituted or substituted in one position with C₁₋₆-alkyl;
R⁸ is H or C₁₋₆-alkyl; and
X is O, S, NH, CH—NO₂, or NCN.

2. The compound according to claim 1 wherein R¹ and R² are methyl.

3. The compound according to claim 1 wherein R¹ and R² are linked to form methylene.

4. The compound according to any one of claims 1 to 3 wherein R⁴ is H.

5. The compound according to any one of claims 1 to 3 wherein R⁸ is H.

6. The compound according to any one of claims 1 to 3 wherein X is S.

7. The compound according to claim 6 wherein R³ is H; methyl; ethyl; n-propyl; isobutyl; pentyl; tert-butoxycarbonyl; ethoxycarbonylmethyl; ethoxycarbonylcyclopropylmethyl; benzyl, which may be unsubstituted or independently substituted in one or two positions with trifluoromethyl, methoxy, trifluoromethoxy, and nitro; phenylethyl; phenylpropyl; furylmethyl; methyl-furylmethyl; thienylmethyl; methyl-pyrrolylmethyl; pyridylmethyl; methyl-1H-imidazolylmethyl; or quinolinylmethyl.

8. The compound according to claim 6 wherein R⁵ is H or methyl.

9. The compound according to claim 6 wherein R⁶ is H.

10. The compound according to claim 6, wherein R⁷ is ethyl, n-butyl, isobutyl, sec-butyl, tert-butyl, hexyl, cyclopropyl, cyclopentyl, cyclopropylmethyl, allyl, methoxypropyl, morpholinylethyl, furylmethyl, benzyl, phenylethyl, fluorobenzyl, or methoxybenzyl.

11. The compound according to any one of claims 1 to 3 wherein X is oxygen.

12. The compound according to claim 11 wherein R³ is methyl or benzyl.

13. The compound according to claim 11 wherein R⁵ is H.

14. The compound according to claim 11 wherein R⁶ is H or methyl.

15. The compound according to claim 11 wherein R⁷ is ethyl, allyl, benzyl, ethoxycarbonylethyl, piperazinylethyl, piperazinylpropyl, methylbenzyl, fluorobenzyl, bromobenzyl, dichlorobenzyl, methoxybenzyl, trifluoromethylbenzyl, phenylethyl, thienylethyl, pyridinylmethyl, methylpyrazinylmethyl, or 4-methylpiperazin-1-yl.

16. The compound according to claim 11 wherein R⁶ and R⁷ together with the nitrogen to which they are attached form a piperazine, which may be unsubstituted or substituted in one position with methyl.

17. The compound according to any one of claims 1 to 3 wherein X is NH.

18. The compound according to claim 17 wherein R³ is methyl.

19. The compound according to claim 17 wherein R⁵ and R⁶ both are H.

20. The compound according to claim 17 wherein R⁷ is n-butyl, n-pentyl, or benzyl.

21. The compound according to claim 1, which is a compound selected from the group of:
N-[(3aS*,6S*,7aS*)-1-benzyl-3a-(3,4-dimethoxyphenyl)octahydro-1H-indol-6-yl]-N′-ethylthiourea,
N-[(3aS*,6R*,7aS*)-1-benzyl-3a-(3,4-dimethoxyphenyl)octahydro-1H-indol-6-yl]-N′-ethylthiourea,
N-[(3aS*,6R*,7aS*)-1-benzyl-3a-(3,4-dimethoxyphenyl)octahydro-1H-indol-6-yl]-N′-butylthiourea,
N-[(3aS*,6S*,7aS*)-1-benzyl-3a-(3,4-dimethoxyphenyl)octahydro-1H-indol-6-yl]-N′-butylthiourea,
N-benzyl-N′-[(3aS*,6R*,7aS*)-1-benzyl-3a-(3,4-dimethoxyphenyl)octahydro-1H-indol-6-yl]thiourea,
N-benzyl-N′-[(3aS*,6S*,7aS*)-1-benzyl-3a-(3,4-dimethoxyphenyl)octahydro-1H-indol-6-yl]thiourea,
N-[(3aS*,6S*,7aS*)-1-benzyl-3a-(3,4-dimethoxyphenyl)octahydro-1H-indol-6-yl]-N′-ethylurea,
tert-butyl (3aS*,6R*,7aS*)-6-{[(benzylamino)carbonothioyl]amino}-3a-(3,4-dimethoxyphenyl)octahydro-1H-indole-1-carboxylate,
tert-butyl (3aS*,6S*,7aS*)-6-{[(benzylamino)carbonothioyl]amino}-3a-(3,4-dimethoxyphenyl)octahydro-1H-indole-1-carboxylate,
N-benzyl-N′-[(3aS*,6R*,7aS*)-3a-(3,4-dimethoxyphenyl)octahydro-1H-indol-6-yl]thiourea,
N-benzyl-N′-[(3aS*,6R*,7aS*)-3a-(3,4-dimethoxyphenyl)-1-(quinolin-3-ylmethyl)octahydro-1H-indol-6-yl]thiourea,
N-benzyl-N′-{(3 aS*,6R*,7aS*)-3a-(3,4-dimethoxyphenyl)-1-[3-(trifluoromethyl)benzyl]octahydro-1H-indol-6-yl}thiourea,
N-benzyl-N′-{(3 aS*,6R*,7aS*)-3a-(3,4-dimethoxyphenyl)-1-[4-(trifluoromethoxy)benzyl]octahydro-1H-indol-6-yl}thiourea,
N-benzyl-N′-[(3 aS*,6R*,7aS*)-3a-(3,4-dimethoxyphenyl)-1-(3-phenylpropyl)octahydro-1H-indol-6-yl]thiourea,
N-benzyl-N′-[(3aS*,6R*,7aS*)-3a-(3,4-dimethoxyphenyl)-1-(pyridin-3-ylmethyl)octahydro-1H-indol-6-yl]thiourea,
N-benzyl-N′-[(3aS*,6R*,7aS*)-3a-(3,4-dimethoxyphenyl)-1-(4-methoxybenzyl)octahydro-1H-indol-6-yl]thiourea,
ethyl [(3aS*,6R*,7aS*)-6-{[(benzylamino)carbonothioyl]amino}-3a-(3,4-dimethoxyphenyl)octahydro-1H-indol-1-yl]acetate,
N-benzyl-N′-[(3 aS*,6R*,7aS*)-3a-(3,4-dimethoxyphenyl)-1-(4-nitrobenzyl)octahydro-1H-indol-6-yl]thiourea,
N-benzyl-N′-[(3aS*,6R*,7aS*)-3a-(3,4-dimethoxyphenyl)-1-pentyloctahydro-1H-indol-6-yl]thiourea,
N-benzyl-N′-[(3aS*,6S*,7aS*)-3a-(3,4-dimethoxyphenyl)octahydro-1H-indol-6-yl]thiourea,
N-allyl-N′-[(3aS*,6R*,7aS*)-1-benzyl-3a-(3,4-dimethoxyphenyl)octahydro-1H-indol-6-yl]urea trifluoroacetate,
N-[(3aS*,6R*,7aS*)-1-benzyl-3a-(3,4-dimethoxyphenyl)octahydro-1H-indol-6-yl]-N′-(2-furylmethyl)thiourea trifluoroacetate,
N-[(3aS*,6R*,7aS*)-1-benzyl-3a-(3,4-dimethoxyphenyl)octahydro-1H-indol-6-yl]-N′-(2-morpholin-4-ylethyl)thiourea trifluoroacetate,
N-benzyl-N′-[(3aS*,6R*,7aS*)-1-benzyl-3a-(3,4-dimethoxyphenyl)octahydro-1H-indol-6-yl]urea trifluoroacetate,
N-[(3aS*,6R*,7aS*)-1-benzyl-3a-(3,4-dimethoxyphenyl)octahydro-1H-indol-6-yl]-N′-(3-methoxypropyl)thiourea trifluoroacetate,
N-[(3aS*,6R*,7aS*)-1-benzyl-3a-(3,4-dimethoxyphenyl)octahydro-1H-indol-6-yl]-N′-(cyclopropylmethyl)thiourea trifluoroacetate,
N-allyl-N′-[(3aS*,6R*,7aS*)-1-benzyl-3a-(3,4-dimethoxyphenyl)octahydro-1H-indol-6-yl]thiourea,
N-[(3aS*,6R*,7aS*)-1-benzyl-3a-(3,4-dimethoxyphenyl)octahydro-1H-indol-6-yl]-N′-isobutylthiourea trifluoroacetate,
N-[(3aS*,6R*,7aS*)-1-benzyl-3a-(3,4-dimethoxyphenyl)octahydro-1H-indol-6-yl]-N′-(2-phenylethyl)thiourea trifluoroacetate,
ethyl N-({[(3aS*,6R*,7aS*)-1-benzyl-3a-(3,4-dimethoxyphenyl)octahydro-1H-indol-6-yl]amino}carbonyl)-beta-alaninate trifluoroacetate,
N-butyl-N′-[(3aS*,6R*,7aS*)-3a-(3,4-dimethoxyphenyl)-1-ethyloctahydro-1H-indol-6-yl]thiourea trifluoroacetate,
N-butyl-N′-[(3aS*,6R*,7aS*)-3a-(3,4-dimethoxyphenyl)-1-propyloctahydro-1H-indol-6-yl]thiourea trifluoroacetate,
N-butyl-N′-[(3aS*,6R*,7aS*)-3a-(3,4-dimethoxyphenyl)-1-isobutyloctahydro-1H-indol-6-yl]thiourea trifluoroacetate,
N-butyl-N′-[(3aS*,6R*,7aS*)-3a-(3,4-dimethoxyphenyl)-1-pentyloctahydro-1H-indol-6-yl]thiourea trifluoroacetate,
N-butyl-N′-[(3aS*,6R*,7aS*)-3a-(3,4-dimethoxyphenyl)-1-(2-phenylethyl)octahydro-1H-indol-6-yl]thiourea trifluoroacetate,
ethyl 2-{[(3aS*,6R*,7aS*)-6-{[(butylamino)carbonothioyl]amino}-3a-(3,4-dimethoxyphenyl)octahydro-1H-indol-1-yl]methyl}cyclopropanecarboxylate trifluoroacetate,
N-butyl-N′-[(3 aS*,6R*,7aS*)-3a-(3,4-dimethoxyphenyl)-1-(3-furylmethyl)octahydro-1H-indol-6-yl]thiourea trifluoroacetate,
N-butyl-N′-{(3aS*,6R*,7aS*)-3a-(3,4-dimethoxyphenyl)-1-[(1-methyl-1H-pyrrol-2-yl)methyl]octahydro-1H-indol-6-yl}thiourea trifluoroacetate,
N-butyl-N′-{(3aS*,6R*,7aS*)-3a-(3,4-dimethoxyphenyl)-1-[(5-methyl-2-furyl)methyl]octahydro-1H-indol-6-yl}thiourea trifluoroacetate,
N-butyl-N′-[(3aS*,6R*,7aS*)-3a-(3,4-dimethoxyphenyl)-1-(3-thienylmethyl)octahydro-1H-indol-6-yl]thiourea trifluoroacetate,
N-butyl-N′-{(3aS*,6R*,7aS*)-3a-(3,4-dimethoxyphenyl)-1-[(4-methyl-1H-imidazol-5-yl)methyl]octahydro-1H-indol-6-yl}thiourea trifluoroacetate,
N-butyl-N′-[(3aS*,6R*,7aS*)-3a-(3,4-dimethoxyphenyl)-1-(3-phenylpropyl)octahydro-1H-indol-6-yl]thiourea trifluoroacetate,
N-[(3aS*,6R*,7aS*)-1-benzyl-3a-(3,4-dimethoxyphenyl)octahydro-1H-indol-6-yl]-N′-(pyridin-4-ylmethyl)urea trifluoroacetate,
N′-[(3 aS*,6R*,7aS*)-1-benzyl-3a-(3,4-dimethoxyphenyl)octahydro-1H-indol-6-yl]-N-ethyl-N-methylurea trifluoroacetate,
N-[(3aS*,6R*,7aS*)-3a-(3,4-dimethoxyphenyl)-1-methyloctahydro-1H-indol-6-yl]-N′-(4-fluorobenzyl)thiourea trifluoroacetate,
N-[(3aS*,6R*,7aS*)-3a-(3,4-dimethoxyphenyl)-1-methyloctahydro-1H-indol-6-yl]-N′-[2-(2-thienyl)ethyl]urea trifluoroacetate,
N-[(3aS*,6R*,7aS*)-3a-(3,4-dimethoxyphenyl)-1-methyloctahydro-1H-indol-6-yl]-N′-(2-phenylethyl)urea trifluoroacetate,
N-[(3aS*,6R*,7aS*)-3a-(3,4-dimethoxyphenyl)-1-methyloctahydro-1H-indol-6-yl]-N′-(4-methoxybenzyl)thiourea trifluoroacetate,
N-allyl-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-hexylthiourea trifluoroacetate,
N-[(3aS*,6R*,7aS*)-3a-(3,4-dimethoxyphenyl)-1-methyloctahydro-1H-indol-6-yl]-N′-(3-methylbenzyl)urea trifluoroacetate,
N-[(3aS*,6R*,7aS*)-3a-(3,4-dimethoxyphenyl)-1-methyloctahydro-1H-indol-6-yl]-N′-(4-methoxybenzyl)urea trifluoroacetate,
N-benzyl-N′-[(3aS*,6S*,7aS*)-3a-(3,4-dimethoxyphenyl)-1-methyloctahydro-1H-indol-6-yl]guanidine trifluoroacetate,
N-butyl-N′-[(3 aS*,6S*,7aS*)-3a-(3,4-dimethoxyphenyl)-1-methyloctahydro-1H-indol-6-yl]guanidine trifluoroacetate,
N-[(3aS*,6S*,7aS*)-3a-(3,4-dimethoxyphenyl)-1-methyloctahydro-1H-indol-6-yl]-N′-pentylguanidine trifluoroacetate,
N-butyl-N′-[(3aS*,6R*,7aS*)-3a-(3,4-dimethoxyphenyl)-1-methyloctahydro-1H-indol-6-yl]guanidine trifluoroacetate,
N-[(3 aS*,6R*,7aS*)-3a-(3,4-dimethoxyphenyl)-1-methyloctahydro-1H-indol-6-yl]-N′-[4-(trifluoromethyl)benzyl]urea trifluoroacetate,
N-(2,4-dichlorobenzyl)-N′-[(3aS*,6R*,7aS*)-3a-(3,4-dimethoxyphenyl)-1-methyloctahydro-1H-indol-6-yl]urea trifluoroacetate,
N-[(3aS*,6R*,7aS*)-3a-(3,4-dimethoxyphenyl)-1-methyloctahydro-1H-indol-6-yl]-N′-(4-fluorobenzyl)urea trifluoroacetate,
N-(4-bromobenzyl)-N′-[(3aS*,6R*,7aS*)-3a-(3,4-dimethoxyphenyl)-1-methyloctahydro-1H-indol-6-yl]urea trifluoroacetate,
N′-butyl-N-[(3aS*,6R*,7aS*)-3a-(3,4-dimethoxyphenyl)-1-methyloctahydro-1H-indol-6-yl]-N-methylthiourea trifluoroacetate,
N′-butyl-N-[(3aS*,6S*,7aS*)-3a-(3,4-dimethoxyphenyl)-1-methyloctahydro-1H-indol-6-yl]-N-methylthiourea trifluoroacetate,
N′-benzyl-N-[(3aS*,6S*,7aS*)-3a-(3,4-dimethoxyphenyl)-1-methyloctahydro-1H-indol-6-yl]-N-methylthiourea,
N′-benzyl-N-[(3aS*,6S*,7aS*)-3a-(3,4-dimethoxyphenyl)-1-methyloctahydro-1H-indol-6-yl]-N-methylthiourea trifluoroacetate,
N-[(3aS*,6S*,7aS*)-1-benzyl-3a-(3,4-dimethoxyphenyl)octahydro-1H-indol-6-yl]-4-methylpiperazine-1-carboxamide bis(trifluoroacetate),
N-[(3aS*,6R*,7aS*)-3a-(3,4-dimethoxyphenyl)-1-methyloctahydro-1H-indol-6-yl]-4-methylpiperazine-1-carboxamide bis(trifluoroacetate),
N-[(3aS*,6R*,7aS*)-3a-(3,4-dimethoxyphenyl)-1-methyloctahydro-1H-indol-6-yl]piperazine-1-carboxamide, bis(trifluoroacetate),
N-[(3aS*,6R*,7aS*)-3a-(3,4-dimethoxyphenyl)-1-methyloctahydro-1H-indol-6-yl]-N′-(2-piperazin-1-ylethyl)urea, tris(trifluoroacetate),
N-[(3aS*,6R*,7aS*)-3a-(3,4-dimethoxyphenyl)-1-methyloctahydro-1H-indol-6-yl]-N′-[3-(4-methylpiperazin-1-yl)propyl]urea, tris(trifluoroacetate),
N-[(3aS*,6R*,7aS*)-3a-(3,4-dimethoxyphenyl)-1-methyloctahydro-1H-indol-6-yl]-N′-[(5-methylpyrazin-2-yl)methyl]urea trifluoroacetate,
N-[(3 aS*,6R*,7aS*)-3a-(3,4-dimethoxyphenyl)-1-methyloctahydro-1H-indol-6-yl]-N′-(4-methylpiperazin-1-yl)urea tris(trifluoroacetate),
N-[(3aS*,6R*,7aS*)-1-benzyl-3a-(3,4-dimethoxyphenyl)octahydro-1H-indol-6-yl]-N′-cyclopropylthiourea trifluoroacetate,
N-[(3aS*,6R*,7aS*)-1-benzyl-3a-(3,4-dimethoxyphenyl)octahydro-1H-indol-6-yl]-N′-(sec-butyl)thiourea trifluoroacetate,
N-[(3 aS*,6R*,7aS*)-1-benzyl-3a-(3,4-dimethoxyphenyl)octahydro-1H-indol-6-yl]-N′-cyclopentylthiourea trifluoroacetate,
N-[(3aS*,6R*,7aS*)-3a-(3,4-dimethoxyphenyl)-1-methyloctahydro-1H-indol-6-yl]-N′-[(1S)-1-phenylethyl]urea trifluoroacetate,
N-allyl-N′-[(3aS*,6S*,7aS*)-3a-(1,3-benzodioxol-5-yl)-1-benzyloctahydro-1H-indol-6-yl]urea hydrochloride,
N-allyl-N′-[(3aS*,6R*,7aS*)-3a-(1,3-benzodioxol-5-yl)-1-benzyloctahydro-1H-indol-6-yl]urea hydrochloride,
N-[(3aS*,6R*,7aS*)-3a-(1,3-benzodioxol-5-yl)-1-benzyloctahydro-1H-indol-6-yl]-N′-ethylthiourea hydrochloride,
N-[(3aS*,6S*,7aS*)-3a-(1,3-benzodioxol-5-yl)-1-benzyloctahydro-1H-indol-6-yl]-N′-ethylthiourea hydrochloride,
N-[(3aS*,6S*,7aS*)-3a-(1,3-benzodioxol-5-yl)-1-benzyloctahydro-1H-indol-6-yl]-N′-benzylthiourea hydrochloride,
N-[(3aS*,6R*,7aS*)-3a-(1,3-benzodioxol-5-yl)-1-benzyloctahydro-1H-indol-6-yl]-N′-butylthiourea hydrochloride,
N-[(3aS*,6R*,7aS*)-3a-(1,3-benzodioxol-5-yl)-1-benzyloctahydro-1H-indol-6-yl]-N′-(tert-butyl)thiourea hydrochloride,
N-[(3 aS*,6S*,7aS*)-3a-(1,3-benzodioxol-5-yl)-1-benzyloctahydro-1H-indol-6-yl]-N′-(tert-butyl)thiourea hydrochloride,
N-[(3aS*,6S*,7aS*)-3a-(1,3-benzodioxol-5-yl)-1-benzyloctahydro-1H-indol-6-yl]-N′-butylthiourea hydrochloride, and
N-[(3aS*,6R*,7aS*)-3a-(1,3-benzodioxol-5-yl)-1-benzyloctahydro-1H-indol-6-yl]-N′-benzylthiourea hydrochloride.

22. A process for the preparation of a compound according to anyone of claims 1 to 3 and 21 comprising at least one of the following steps:
(a) coversion of a nitrile to a cyclopropanecarbonitrile,
(b) reduction of the cyclopropanecarbonitrile to give a cyclopropanecarbaldehyde,
(c) coversion of the cyclopropanecarbaldehyde to an imine and further reaction of the imine with an α,β-unsaturatedketone to form an octahydro-indol-6-ketone,
(d) treatment of the octahydro-indol-6-ketone with ammonia, a first amine or a salt thereof and then reduction to give a second amine, and
(e) reaction of the second amine with an isocyanate, isothiocyanate, triphosgene, carboximidamide, or chloroformate.

23. A process of preparing a compound of the general formula (I): 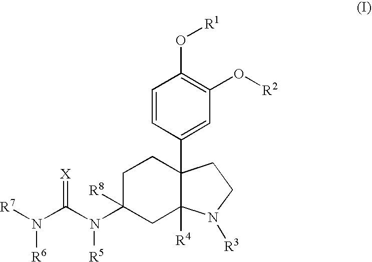
wherein:
R¹ is C₁₋₆-alkyl,
R² is C₁₋₆-alkyl,
or R¹ and R² are linked to form C₁₋₃-alkylene,
R³ is H, C₁₋₆-alkyl, C₁₋₆-alkoxycarbonyl, C₁₋₆-alkoxycarbonyl-C₁₋₆-alkyl, C₁₋₆-alkoxycarbonyl-C₃₋₆-cycloalkyl-C₁₋₆ alkyl, aryl-C₁₋₆-alkyl, heteroaryl-C₁₋₄ alkyl, aryl or heteroaryl, wherein any aryl or heteroaryl may be unsubstituted or independently substituted with C₁₋₆-alkyl, C₁₋₆-alkoxy, C₁₋₆-alkoxycarbonyl, C₁₋₆-alkoxycarbonyl-C₁₋₆-alkyl, C₁₋₆-alkoxycarbonyl-C₃₋₆-cycloalkyl-C₁₋₆ alkyl, halo-C₁₋₆-alkyl, halo-C₁₋₆-alkoxy, or nitro,
R⁴ is H or C₁₋₆-alkyl,
R⁵ is H, OH, C₁₋₆-alkyl, C₁₋₆-alkylaminocarbonyl, or C₁₋₆-alkylaminothioxylcarbonyl,
R⁶ is H or C₁₋₆-alkyl,
R⁷ is H, C₁₋₆-alkyl, C₂₋₆-alkenyl, C₃₋₈-cycloalkyl, C₃₋₈-cycloalkyl-C₁₋₆-alkyl, C₁₋₆-alkoxy, C₁₋₆-alkoxy-C₁₋₆alkyl, C₁₋₆-alkylcarbonyl, C₁₋₆-alkoxycarbonyl, C₁₋₆-alkoxycarbonyl-C₁₋₆-alkyl, heterocyclo-C₁₋₆-alkyl, heteroaryl-C₁₋₆-alkyl, aryl-C₁₋₆-alkyl, arylcarbonyl, heteroarylcarbonyl, or heterocyclyl, wherein any aryl, heteroaryl and heterocyclo may be unsubstituted or substituted in one, two or three positions with C₁₋₆-alkyl, halo-C₁₋₆-alkyl, C₁₋₆-alkoxy, or halogen,
or R⁶ and R⁷ are linked to form C₄₋₆-alkylene or together with the nitrogen atom to which they are attached form a piperazine ring, which may be unsubstituted or substituted in one position with C₁₋₆-alkyl,
R⁸ is H or C₁₋₆-alkyl, and
X is O, S, NH, CH—NO₂, or NCN,
comprising reacting an amine compound of the general formula (II): 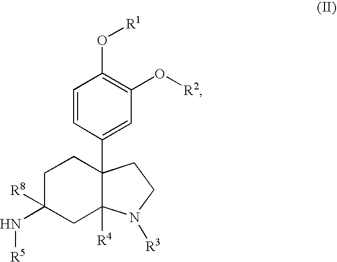
wherein R¹, R², R³, R⁴, R⁵, and R⁸ are defined above,
with a reagent to give the compound of the general formula (I).

24. The method according to claim 23, wherein the reagent is an isocyanate, isothiocyanate, triphosgene, carboximidamide, or chloroformate.

25. A pharmaceutical formulation comprising a compound according to anyone of claims 1 to 3 and 21 as an active ingredient, and a pharmaceutically acceptable diluent or carrier.

26. A pharmaceutical formulation for use in the treatment or prophylaxis of obesity comprising a compound according to anyone of claims 1 to 3 and 21.

27. A method for the treatment or prophylaxis of obesity, diabetes mellitus, hyperlipidemia, hyperglycemia, depression, anxiety, or urinary incontinence, or for modulation of appetite, said method comprising administering to a subject in need of such treatment an effective amount of a compound according to anyone of claims 1 to 3 and 21.

28. A method for the treatment or prophylaxis of a disorder related to the MCH1R receptor, said method comprising administering to a subject in need of such treatment an effective amount of a compound according to anyone of claims 1 to 3 and 21.

29. A method for modulating MCH1R receptor activity, comprising administering to a subject in need thereof an effective amount of a compound according to any one of claims 1 to 3 and 21.
